(19) United States
(12) Patent Application Publication
(10) Pub. No.: US 2023/0329755 A1
(43) Pub. Date: Oct. 19, 2023

(54) BONE FLAP FIXATION DEVICE

(71) Applicant: RevBio, Inc., Lowell, MA (US)

(72) Inventors: Brian J. Hess, Charlestown, MA (US); David J. Kosh, Norton, MA (US); George W. Kay, Sharon, MA (US); Michael C. Brown, Harvard, MA (US); Christy E. Cavaleri, Chelmsford, MA (US); Kevin T. Foley, Germantown, TN (US); Eric J. Woodard, Westwood, MA (US); Jonathan R. Slotkin, Lewisburg, PA (US)

(21) Appl. No.: 18/010,603

(22) PCT Filed: Jun. 15, 2021
(86) PCT No.: PCT/US2021/037466

§ 371 Date: Dec. 15, 2022

Related U.S. Application Data
(60) Provisional application No. 63/114,920, filed on Nov. 17, 2020.
(60) Provisional application No. 63/039,306, filed on Jun. 15, 2020.

(51) Int. Cl. A61B 17/68; A61B 17/80

(52) U.S. Cl. CPC A61B 17/688; A61B 17/809; A61B 2017/00004

(57) ABSTRACT

Described herein are devices for easily and predictably allowing a single user to position fixate a flap or segment of bone of any size in position adjacent to native bone to allow filling of the kerf with an adhesive composition and sealing the kerf, as well as related methods.

### CROSS-REFERENCE TO RELATED APPLICATIONS

This application claims priority to U.S. Provisional Application No. 63/039,306, filed Jun. 15, 2020, and U.S. Provisional Application No. 63/114,920, filed Nov. 17, 2020. The entire contents of each of the foregoing applications is hereby incorporated herein by reference.

### BACKGROUND

Current devices and methods of use related to the repair and fixation of cracks, gaps, fissures, leaks or other defects in bone often lack sealing ability, do not optimize cosmesis, and do not replace the missing void material. These practices often leave the repaired region weak and vulnerable to future repeated damage, particularly when said region is subjected to a wet environment. There is currently a need for devices and methods which hold a bone flap of any shape, size, and/or location in position, such that the flap may be permanently secured to the surrounding bone and the flap fixation device(s) optionally removed following the secure fixation of the flap.

### SUMMARY

The present disclosure features devices for fixating a flap or segment of bone (e.g., damaged bone) of any shape, size, and/or location (such as a flap created by an osteotomy, e.g., a cranial flap) into position, such that the kerf or gap created between the flap or segment of bone and the native bone can be filled with an adhesive composition to fixate said flap or segment of bone, as well as assemblies of devices and related methods of use thereof. Various bone types which are compatible with use of these devices include, but are not limited to, the cranium, spine, arm bones, and leg bones. Said device embodiments can be used on or with bone flaps created surgically, e.g., a craniotomy, or used on fractured or broken bones (e.g., an uneven break). In some embodiments, various devices are used together to hold pieces of a bone together or to provide proper spacing for an adhesive composition to be delivered.

In one aspect, the present disclosure features a device (e.g., or assembly of devices) useful in the prevention and treatment of, or recovery from, a disease or disorder in a subject. In some embodiments, the disease or disorder comprises a bone disease or disorder, e.g., cancer (e.g., osteosarcoma), osteoporosis, rickets, osteogenesis imperfecta, Paget’s disease of the bone, hearing loss, renal osteodystrophy, a malignancy of the bone, infection of the bone, severe and handicapping malocclusion, osteonecrosis, or other genetic or developmental disease. In some embodiments, the device (e.g., or assembly of devices) is used to regenerate bone in a defect caused by a disease or condition, alone or together with an adhesive composition. In some cases, the subject has experienced a trauma, such as a broken bone, or fractured bone relating to a disease or condition. The devices described herein may be used to treat a subject suffering from or afflicted with any disease, condition, injury or procedure that impacts the structural integrity of the bony skeleton. In some embodiments, the subject is a child. In some embodiments, the subject is an adult. In some embodiments, the subject is a senior (e.g., an adult over the age of about 50, about 55, about 60, about 65, about 70, about 75, about 80) or in a decline of the skeletal state. In some embodiments, the subject is a human or a non-human vertebrate.

The present disclosure also features compositions of adhesive compositions and related methods of use and means for packaging and delivery of said adhesive compositions. In some embodiments, said adhesive composition comprises a biomaterial or combination of biomaterials. In some embodiments, said biomaterial comprises a bone substitute. Said adhesive composition can be introduced into or onto the subject (e.g., at the surgical site) by means of a single injection, multiple injections, or other means of application. In an embodiment, the adhesive composition fixates the flap in position and optionally eliminates the need for conventional hardware fixation devices (e.g., plates/screws, bur hole covers, clips) which are either permanent or require a subsequent surgical intervention to remove. The device of the present disclosure may allow a user to easily and predictably position and temporarily fixate a flap in position to allow filling of the kerf with an adhesive composition. Said devices may be comprised of a biocompatible material. Said biocompatible material may comprise organic or synthetic matter or a combination thereof. Said devices may also be used in conjunction with any adhesive composition, e.g., bone substitute, e.g., a calcium phosphate bone cement or a polymethyl methacrylate (PMMA) bone cement. The bone flap fixation devices described herein may be removed prior to closure of the flap or may be retained at the surgical site for an extended length of time (e.g., minutes, hours, days, weeks, months, years after application, or indefinitely). In some embodiments, the flap may be of any size, e.g., from 1 millimeter (mm) to 50 centimeters (cm).

The device (e.g., or assembly of devices) may utilize mechanical and/or chemical means for fixating the flap in position (e.g., the original position of the flap, or an alternate position of the flap). In some embodiments, the device utilizes mechanical bonding features such as screws, pins, plates, protrusions, clamps, wedges, or additional supportive structures. In an embodiment, the mechanical bonding features comprise a biocompatible material. In some embodiments, the device comprises a mechanical feature that fits within the dimensions of the kerf. This mechanical feature that fits within the kerf may comprise a structure that bonds to a bone in one or more locations using screws, rods, plates, suction, or adhesive bonding to provide central support and adjustability for positioning the flap in position. Said mechanical feature may comprise a biocompatible material. Said biocompatible material may comprise organic or synthetic matter.

In another aspect, the present disclosure features devices (e.g., or assembly of devices) comprised of biocompatible material. Said biocompatible material may comprise a naturally occurring or non-naturally occurring material (e.g., organic or synthetic matter). For example, said biocompatible material may comprise one or more of: steel or steel alloys; titanium or titanium alloys; plastic materials such as PEEK, Polycarbonate, PET, PE, polypropylene; xenograft; allograft; bone substitute; similar such materials and combinations thereof. The devices (e.g., or assembly of devices) featured in the present disclosure may comprise a resorbable biocompatible material that may be dispersed. In some embodiments, the resorbable biocompatible material is replaced with host tissue over time. In some embodiments, the device comprises a non-resorbable material, such as metal, plastic or other non-bone material. In some embodiments, the non-resorbable material is of sufficient strength to withstand placement, adjustment, and removal without compromising the position of the flap. In some embodiment, the device may comprise a malleable material, e.g., to allow manual adjustment of flap alignment into position.

Additional features of the present disclosure and the devices, assemblies of devices, compositions, and methods of use thereof are described herein in the Detailed Description, Figures and Claims.

### BRIEF DESCRIPTION OF THE FIGURES

FIG. 1 depicts an exemplary assembly of an exemplary flap kerf intraoperative spacer device.

FIG. 2 depicts delivery of an adhesive composition while multiple exemplary spacer devices are in place between the flap and the native bone, such that the flap may be fixated in position.

FIG. 3 is an image depicting an exemplary flap intraoperative retainer connected to the flap and the native bone such that the flap stays in place.

FIG. 4 depicts an exemplary flap intraoperative retainer device and spacer devices in place while an adhesive composition is delivered.

FIGS. 5A-D depict exemplary flap retainers. FIG. 5A depicts an exemplary flap retainer with relief device from a top-down perspective; FIG. 5B depicts an exemplary flap retainer with relief device from a bottom-up perspective; FIG. 5C depicts an exemplary flap retainer with relief device with additional protrusions for fixation; and FIG. 5D is a depiction of an exemplary flap retainer with relief and exposing further exemplary protrusions for fixation.

FIGS. 6A-D depict a method of use of an exemplary flap retainer with relief utilizing screws for fixation of the flap.

FIGS. 7A-B depict an exemplary use of a flap retainer with relief utilizing protrusions for fixation of the flap.

FIG. 8 depicts multiple exemplary flap retainers with reliefs utilizing screws for fixation spaced evenly to fixate the flap and native bone, in use with an adhesive composition delivery device.

FIG. 9 depicts of a close-up view of multiple exemplary flat retainers with relief devices utilizing protrusions spaced evenly to fixate the flap and native bone, in use with an adhesive composition delivery device.

FIG. 10 depicts an exemplary rotating CF fixation support, and particularly the rotational use of the support device.

FIG. 11 depicts an exemplary rotating CF fixation support with the foot in a closed position vs with the foot in an open and extended position, as well as the rotating motion of the foot.

FIG. 12 is a depiction of multiple exemplary rotating CF fixation supports being utilized in the kerf for fixation of the flap.

FIG. 13 depicts two close-up views of an exemplary rotating CF fixation support with a dual foot assembly in use in the kerf, specifically how the foot assembly locks in place.

FIG. 14 is a depiction of an exemplary flap compressive support screw device.

FIG. 15 is a close-up, side view of an exemplary flap compressive support screw device.

FIG. 16 is a depiction of multiple exemplary flap compressive support screws in use in the kerf to secure the flap in position, and delivery of an adhesive composition.

FIG. 17 is a depiction of an exemplary bone flap articulating arm device utilizing a primary alignment protrusion, fixating the flap in position and a clamp support attached to the other end of the device to allow manipulation of the device.

FIG. 18 is a close-up view of an exemplary flap articulating arm device in use fixating the flap while the kerf is filled with an adhesive composition delivery device.

FIG. 19 is a depiction of an exemplary kerf flap (KF) fixation device comprising a rubber footing utilizing compression force for a locking feature.

FIG. 20 is an alternative angle view of an exemplary kerf flap fixation device.

FIG. 21 is a view of multiple exemplary kerf flap fixation devices fixating the flap in position.

FIG. 22 is an angular view of multiple exemplary kerf flap fixation devices in use with a locking mechanism to hold device position.

FIG. 23 is an overhead depiction of multiple exemplary kerf flap fixation devices in use in a locked position.

FIGS. 24A-B depicts exemplary flap step fixation devices. FIG. 24A illustrates an exemplary flap step fixation device. FIG. 24B depicts a cross-sectional view of an exemplary flap step fixation device and an overhead view of the same.

FIG. 25 is a depiction of multiple exemplary flap step fixation devices in use in the kerf fixating the flap in use with a composition delivery device, as well as a close-up view of delivery of an adhesive composition around the device.

FIG. 26 depicts multiple views of an exemplary bone flap alignment wedge device.

FIG. 27 depicts multiple exemplary bone flap alignment wedge devices in use holding a flap in position while an adhesive composition is delivered.

FIG. 28 is a cross sectional view of an exemplary bone flap alignment wedge in use aligning the native bone and the bone flap.

FIG. 29 depicts an exemplary rotating fixation support device.

FIG. 30 depicts multiple views of an exemplary rotating fixation support device, with the foot in the retracted and closed position, with the foot in the open position, and an overhead view.

FIG. 31 depicts multiple exemplary rotating fixation support devices in use holding a flap in position.

FIG. 32 depicts two close-up views of an exemplary rotating fixation support with a single-foot assembly in use in the kerf, specifically how the foot assembly locks in place.

FIG. 33 depicts an exemplary CF fixation support device.

FIG. 34 depicts multiple exemplary iterations of the CF fixation support device.

FIG. 35 depicts exemplary devices of various sizes and shapes with a different amount of burr holes.

FIG. 36 depicts the progression of the exemplary kerf flap fixation device.

### DETAILED DESCRIPTION

The present disclosure features, inter alia, a device for fixating a flap or segment of bone of any shape, size, and/or location in position and an adhesive composition, as well as assemblies of devices and related methods of using the same. Components and embodiments of the devices, assemblies, compositions, and methods of use thereof are described herein in greater detail. Aspects of one embodiment may be found in all embodiments, or the devices may be used in concert (e.g., as an assembly of devices), even if not explicitly described.

As used herein, the term “device” refers to the mechanical apparatus or chemical composition used to fixate a flap or segment of bone, e.g., in position. In an embodiment, the device fixates the flap or segment of bone so that an adhesive composition may be delivered to the site. In an embodiment, the device is temporarily mounted or attached to the bone, e.g., at least until the adhesive composition hardens to provide a seal. In an embodiment, the device is part of an assembly of devices, comprising, e.g., 2, 3, 4, 5, 6, 7, 8, or more devices. The device may pierce the bone or tissue of the site of use, or the device may instead exert a force on the bone or tissue at the site of use without piercing the bone or tissue. Exemplary devices are described herein and in the Figures.

As used herein, the term “flap” refers to the portion of bone or bone substitute which is repositioned and subsequently fixated into position. In an embodiment, the flap is a cranial flap. In an embodiment, the flap is between about 0.5 mm to about 500 cm along one dimension, e.g., about 0.5 cm to about 250 cm, about 0.5 cm to about 100 cm, 0.5 cm to about 50 cm, 0.5 cm to about 25 cm, or 0.5 cm to about 10 cm.

As used herein, the term “ker” refers to the cut line or gap created by a surgical procedure (e.g., an osteotomy, e.g., a craniotomy).

As used herein, the term “bone substitute” refers to any material other than natural bone. The composition of a bone substitute may comprise a biomaterial, a tissue from a xenograft, allograft, or autograft, or any combination thereof. In an embodiment, the bone substitute is an adhesive composition (e.g., an adhesive composition described herein).

### Devices

The device, device assemblies, and embodiments disclosed herein present a significant innovation over devices in the art. Some embodiments disclosed herein allow for intraoperative or provisional fixation of a flap to provide temporary immobilization; wherein other embodiments allow for definitive fixation of a flap. Intraoperative or provisional fixation of a flap may provide temporary immobilization and flap fixation in its original or corrected position, whereas definitive fixation may provide for complete fixation and delivery of an adhesive composition and wherein the device may remain in place. Further, the delivery of said adhesive composition may be a partial fill or a full fill. In some embodiments, a partial fill enables fixation of the flap. In some embodiments, a full fill enables sealing of the kerf space with a depth equaling the kerf space thickness and circumference equaling the run of the osteotomy, accounting for any burr holes or other surgically created holes. In some embodiments, fixation of the flap allows for delivery of an adhesive composition. In some embodiments, said adhesive composition comprises a biomaterial or combination or biomaterials.

In one aspect, the present disclosure features devices comprising biocompatible material. Said biocompatible material may comprise organic or synthetic matter. For example, said biocompatible material may comprise one or more of: steel or steel alloys; titanium or titanium alloys; plastic materials such as polyetheretherketone (PEEK), polycarbonate, polyethylene terephthalate (PET), polyethylene (PE), polypropylene; xenograft; allograft; similar such materials and combinations thereof. In some embodiments, said devices comprise a resorbable biocompatible material that disperses, e.g., a bone substitute. In some embodiments, said resorbable biocompatible material is replaced with host tissue over time. In some embodiments, devices are removed after intraoperative use, i.e., once the adhesive composition is delivered and has sufficient strength for definitive flap fixation. In some embodiments, flap fixation with said adhesive composition is provided within the kerf space and therefore does not protrude above the kerf space, effectively providing enhanced cosmesis for the patient. In some embodiments, the method comprises applying a sufficient amount of adhesive composition into the kerf space of an osteotomy, effectively providing a seal to prevent cerebral spinal fluid (CSF) leakage through the bone. In some embodiments, the devices described herein are not removed, therefore lowering the risk of infection.

Other devices in the art currently used for flap fixation, e.g., plates and screws, may span across and above the kerf line to fixate the flap to the native bone and are often made from permanent or non-bioresorbable materials, e.g., metal and plastic. For these devices, the kerf space that results from creation of the flap (e.g., craniotomy) is typically left empty unless filled with an adhesive composition, e.g., a bone substitute. To enable fixation between the flap and host bone, these devices may require fixation means, e.g., screws to hold the plate or plates to the native bone (e.g., cranium) for sufficient time after surgery to facilitate flap integration and healing. As a result of their placement position, these devices protrude above the contour of the outer bone surface. In an embodiment, the presence of these devices can be felt by the subject and/or are visible through the soft tissue envelope. These devices are typically left in place for the life of the subject unless a secondary surgery is indicated to remove them. Given that these devices span above and across the kerf space, they do not fill the kerf space and therefore do not provide a watertight seal to prevent fluid leakage (e.g., CSF leakage) through the bone. In contrast, the present disclosure features device embodiments and methods with which to provide a means for flap fixation and kerf sealing without the need for screws or device protrusion beyond the kerf. The present disclosure further features devices comprised of resorbable biocompatible material which may be replaced by host tissue, leaving the osteotomy stronger than it would be with a foreign replacement. The present disclosure also features an adhesive composition, which may comprise a resorbable bone substitute, and which may be used to fixate the flap and seal the kerf.

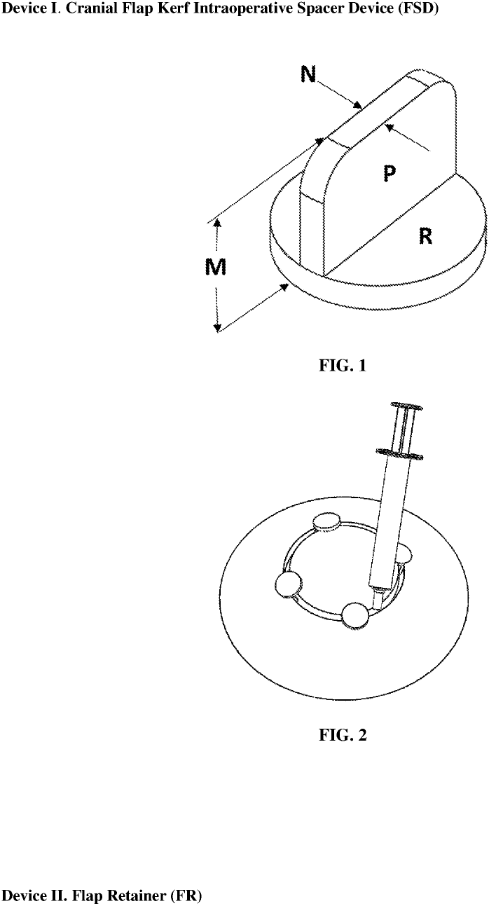

In one aspect, the present disclosure features a flap spacer device, (FSD) (FIG. 1). In some embodiments, an (FSD) may comprise a projection (P) of width n and length m centered on flange (R). Specifically, flange (R) may limit the distance that projection (P) can extend from the exterior of a bone toward the interior of a bone. In some embodiments, width n is substantially the same width as the width of the kerf space, e.g., between 0.25 mm and 10 mm, (between 0.5 mm and 5 mm, 0.5 mm and 2.5 mm, or 1 mm and 5 mm). In some embodiments, length m is substantially the same length as the thickness of the bone in the region of the flap. In some embodiments, length m is between approximately 0.25 mm and 15 mm, e.g., between 0.25 mm and 10 mm, 0.5 mm and 5 mm, or 1 mm and 15 mm. In some embodiments, the length m is not greater than the thickness of the bone in the region of the flap, e.g., 0.25 mm to 15 mm. In some embodiments, the surface of projection (P) may be rough or have protrusions to provide friction between the flap and native bone. In some embodiments, the protrusions are at least 0.05 mm, but less than 1 mm extending from the surface of (P). In some embodiments, the thickness of projection (P) may vary along length m and be substantially thicker towards the flange (R) to serve as a wedge when in use between the flap and native bone. In some embodiments, a plurality of (FSD) devices may be distributed into the kerf space. Said plurality of (FSD) devices may provide uniform spacing for placement of an adhesive composition. In some embodiments, components of (FSD), e.g., flange (R) comprises an adhesive composition, e.g., a resorbable biocompatible material that disperses, e.g., a bone substitute. In some embodiments, the resorbable biocompatible material is replaced with host tissue over time. In some embodiments, (FSD) comprises a non-resorbable material.

In some embodiments, (FSD) comprises a malleable material, e.g., to allow for manual adjustment and alignment of the flap into its position. In some embodiments, said (FSD) is of sufficient size or strength to withstand placement, adjustment, or removal without compromising the positioning of the flap. In some embodiments, (FSD) exerts force only on the posterior surface of the flap, e.g., force upward versus gravity, rather than exerting any force downward onto the anterior surface of the flap, e.g., force downward with gravity, e.g., wherein a craniotomy has been performed, force downward onto the skull. In some embodiments, (FSD)s may remain in the kerf upon completion of the flap fixation procedure. In some embodiments, (FSD)s are used intraoperatively, e.g., to allow for temporary fixation during administration of an adhesive composition. In some embodiments, (FSD)s are removed intraoperatively once the adhesive composition reaches sufficient strength or rigidity to maintain bone flap fixation. In some embodiments, a portion of (FSD) protrudes from the profile of the flap and native bone to allow for manual access, e.g., to allow for manual adjustment of the device utilizing protrusion (P). In some embodiments, wherein (P) protrudes from the profile of the flap and native bone, (FSD) is able to be manually placed, adjusted, and/or removed as needed without the need for use of a second tool, e.g., a screwdriver or mallet. In some embodiments, (FSD)s may remain in the kerf space during injection and setting of the adhesive composition. In some embodiments, once each device has been removed, the adhesive composition is delivered to the gaps left remaining where the devices had been. In some embodiments, this provides for a complete seal around the kerf space. In some embodiments, (FSD)s remain in the kerf space during the placement of an additional device, e.g., another device described herein. In some embodiments, said (FSD)s weight between about 1 g and 500 g, depending on the material composition and size of the device.

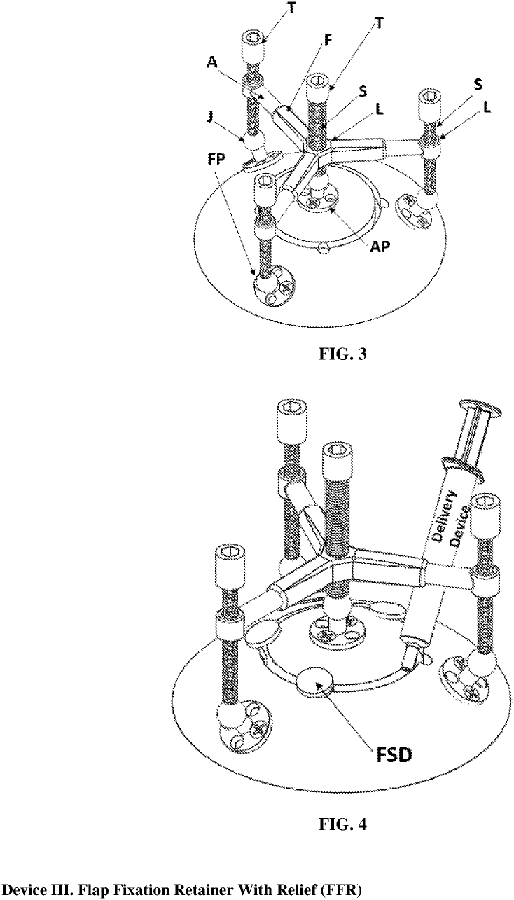

In another aspect, the present disclosure features a flap retainer device (FR) (FIG. 3). In some embodiments, the (FR) may comprise one or more of: a frame (F) comprising a plurality of arms (A) wherein each arm is optionally equipped with a joint articulation (J); a fixed pad (FP); and an adjustable pad (AP). In some embodiments, (FR) also comprises means of affixing the pads (FP) and (AP) to the bone surface, wherein (FP) affixes to the native bone, and (AP) to the flap. In some embodiments, the frame (F) is extendable and adjustable. For example, frame (F) may extend radially, by screw or slide mechanism, to accommodate flaps of different shapes and sizes. In some embodiments, frame (F) comprises a hinged mechanism whereby the angle of the arms relative to one another allows for varying the distance separating fixed pads (FP) to accommodate flaps of different shapes or sizes (FIG. 3). In some embodiments, each arm (A) may feature means to be rigidly extended. In some embodiments, arms (A) may be of an adjustable length and feature means of locking. For example, arms (A) may extend in a direction other than radial to the frame, by screw or slide mechanism, to accommodate greater or lesser surface area of a bone, e.g., the curvature of the skull (FIG. 3). In some embodiments, one or more of the arms (A) is reversibly separable and/or electively separable, e.g., by such mechanism as an elastic snap. In some embodiments adjustable pad (AP) may be rigidly movable relative to the frame (F), e.g., by means of a screw or slide mechanism S, which may be electively locked by mechanism (L). Lock mechanism (L) may be utilized to immobilize the adjustable pad (AP) relative to fixed pads (FP). In some embodiments, such means of affixing fixed pads (FP) and adjustable pad (AP) to the bone surface include screw fixation, adhesive bonding, suction, hook and loop, or other similar such methods. In some embodiments, the (FR) is fixated to the skull and retains the flap in the desired position for placement of an adhesive composition. In some embodiments, said adhesive composition comprises a biomaterial or a combination of biomaterials. In some embodiments, components of (FR), e.g., fixed pad (FP), comprises a resorbable biocompatible material that disperses, e.g., a bone substitute. In some embodiments, the resorbable biocompatible material is replaced with host tissue over time. In some embodiments, (FR) comprises a malleable material, e.g., to allow for manual adjustment and alignment of the flap into position. In some embodiments, said (FR) is of sufficient size or strength to withstand placement, adjustment, or removal without compromising the positioning of the flap. In some embodiments, (FR) exerts force only on the posterior surface of the flap, e.g., force upward versus gravity, rather than exerting any force downward onto the anterior surface of the flap, e.g., force downward with gravity, e.g., wherein a craniotomy has been performed, force downward onto the skull. In some embodiments, (FR) only engages the flap, e.g., it does not exert any force on the native bone. In some embodiments, (FR) is used intraoperatively, e.g., to allow for temporary fixation during administration of an adhesive composition In some embodiments, a portion of (FR) protrudes from the profile of the flap and native bone to allow for manual access, e.g., to allow for manual adjustment of the device. In some embodiments, wherein a portion of (FR) protrudes from the profile of the flap and native bone, (FR) is able to be manually placed, adjusted, and/or removed as needed without the need for use of a second tool, e.g., a screwdriver or mallet. In some embodiments, (FR) is removed intraoperatively, once the adhesive composition reaches sufficient strength or rigidity to maintain bone flap fixation. In some embodiments, once each device has been removed, the adhesive composition is delivered to the gaps left remaining where the devices had been. In some embodiments, this provides for a complete seal around the kerf space. In some embodiments, said devices may remain in the kerf during the placement of additional device, e.g., another device described herein. In some embodiments (FR) weighs between about 10 g and 5000 g, depending on the material composition and size of the device. In some embodiments, the number of holes required in the subject bone is between 1 and 16, depending on user needs.

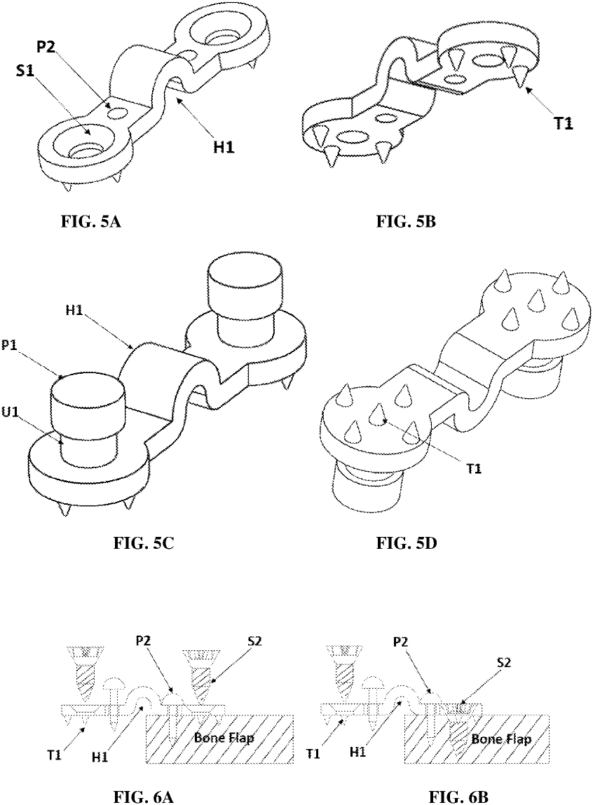

In another aspect, the present disclosure features a flap retainer-with-relief device (FRR). In some embodiments, (FRR) temporarily fixates a flap in its proper alignment utilizing: a kerf line spanning area (H1) allowing continuous delivery and shaping of an adhesive composition; a width sufficient to support a flap but malleable to allow manual adjustment as needed, e.g., 1 mm to 20 mm (e.g., between 1 mm and 10 mm, 5 mm and 15 mm, 10 mm and 20 mm, or 5 mm and 10 mm); a length sufficient to span a distance; and multiple screws (S) which may be placed into native bone to fix the device into position. In some embodiments, (FRR) further comprises a larger screw relief hole (S1) to accommodate larger fixation screws. In some embodiments, (FRR) comprises a series of screw relief holes for greater fixation strength (FIGS. 5A-B). In some embodiments, (FRR) comprises retaining protrusions (T1) which allow said (FRR) to be secured by compressing said (FRR) into native bone using a mallet or other such instrument (FIGS. 5C-D). In some embodiments (FRR) may further comprise two holes on the flap side, the hole located furthest from the relief feature to be used to drill the retaining hole, and the second hole containing the push-on retainer (P1). The first hole may be spaced from the second such that the relief feature will be offset from the edge of the bone flap, and the push on retainer (P1) may be located in the second hole for ready-for-use delivery of an adhesive composition into the hole that was drilled. In some embodiments, (FRR) exerts force only on the posterior surface of the flap, e.g., force upward versus gravity, rather than exerting any force downward onto the anterior surface of the flap, e.g., force downward with gravity, e.g., wherein a craniotomy has been performed, force downward onto the skull. In some embodiments, (FRR) only engages the flap, e.g., it does not exert any force on the native bone. In some embodiments, several such (FRR)s are be spaced out over the run of the osteotomy to provide uniform spacing and sufficient strength for placement of an adhesive composition. In some embodiments, said adhesive composition is comprised of a biomaterial or a combination of biomaterials. In some embodiments, components of (FRR), e.g., retainer (P1), are comprised of an adhesive composition, e.g., a resorbable biocompatible material that disperses, e.g., a bone substitute. In some embodiments, said resorbable biocompatible material is replaced with host tissue over time.

In some embodiments, the (FRR) comprises a malleable material, e.g., to allow for manual adjustment and alignment of the flap into its original or corrected position. In some embodiments, said (FRR)s are of sufficient size or strength to withstand placement, adjustment, and removal without compromising the flap’s position. In some embodiments, multiple (FRR)s may be used for provisional fixation. In some embodiments several such (FRR)s may remain in the kerf upon completion of the flap fixation procedure. In some embodiments (FRR)s are used intraoperatively to allow for temporary fixation during administration of an adhesive composition. In some embodiments, a portion of (FRR), e.g., protrusion (T1), protrudes from the profile of the flap and native bone to allow for manual access, e.g., to allow for manual adjustment of the device utilizing protrusion (T1). In some embodiments, wherein (T1) protrudes from the profile of the flap and native bone, (FRR) is able to be manually placed, adjusted, and/or removed as needed without the need for use of a second tool, e.g., a screwdriver or mallet. In some embodiments, (FRR)s are be removed intraoperatively once the adhesive composition reaches sufficient strength or rigidity to maintain bone flap fixation. In some embodiments (FRR)s remain in the kerf during injection and setting of the adhesive composition. In some embodiments, once each device has been removed, the adhesive composition is delivered to the gaps left remaining where the devices had been. In some embodiments, this provides for a complete seal around the kerf space. In some embodiments, (FRR)s remain in the kerf during the placement of another device, e.g., another device described herein. In some embodiments, an exemplary (FRR) weighs between about 10 g and about 1000 g, depending on the material composition and size of the device. In some embodiments, the number of holes required in the subject bone is between 1 and 4, depending on user needs.

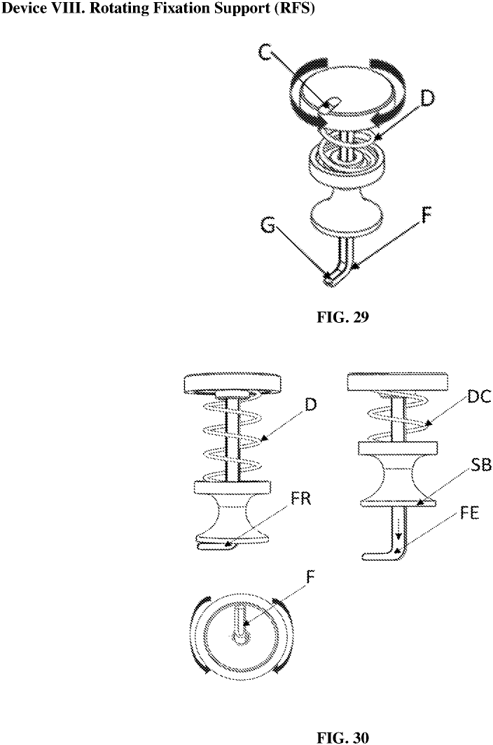

In another aspect, the present disclosure features a rotating flap fixation support device (RFS) which comprises a rotating foot assembly. Said rotating foot assembly may temporarily align or affix a flap in proper alignment utilizing compression forces to fixate the flap in position or tangent contact with a mating surface at time of flap placement. Specifically, said rotating foot assembly utilizes a compression spring to provide upward force towards the outer flap once released to align and fixate the flap in position (FIGS. 29-31). In some embodiments, the rotating foot (F) assembly temporarily affixes to a flap and provides proper alignment utilizing circular base (SB) contact surface with the flap and native bone at time of placement. In some embodiments, the width of the foot (F) is sufficient to allow support of a flap but adjustable to allow manual adjustment if needed, e.g., 0.1 mm to 2 mm (e.g., 0.5 mm to 2 mm, 1 mm to 2 mm, or 0.1 mm to 1 mm). In some embodiments, the device utilizes a thumb handle (H) with alignment markers (C,E) that are coincident with the retaining foot as an aid to properly fixate the flap. In some embodiments, the foot (F) may rotate up to 90 degrees from the cut plane of the flap insert location. In some embodiments, the alignment marker (C,E) allows for orientation of the foot (F) when rotating under compressive load from the (J) position to the (K) position (FIG. 32). In some embodiments, (RFS) comprises a locking feature that allows for rotating travel to be no greater than 90 degree from flap cut plane (FIG. 30). In some embodiments, the device may comprise a friction washer (B) that allows for ease of rotation of the device and which limits rotating motion of the soft durometer compression washer. In some embodiments, (RFS) comprises an array of different durometer compression washers (D) for differing flap retainment strengths. In some embodiments, (RFS) comprises a captured compression spring (D) with a circular base (SB) to create a compressive load to fixate or locate the flap. In some embodiments, spring (D) has a width of about 0.01 mm to about 2 mm (e.g., 0.01 mm-0.1 mm, 0.5 mm-1 mm, 0.05 mm-0.75 mm). In some embodiments, spring (D) has a length of approximately 10 mm to approximately 40 mm (e.g., 10 mm-30 mm, 15 mm-35 mm, 20 mm-40 mm). In some embodiments, spring (D) has a diameter of approximately 3 mm to approximately 20 mm (e.g., 3 mm-10 mm, 5 mm-15 mm, 7 mm-20 mm). In some embodiments, spring (D) comprises approximately 4 active coils (e.g., 3 active coils, 4 active coils, or 5 active coils), and further comprises approximately 6 total coils (e.g., 4 total coils, 5 total coils, 6 total coils, or 7 total coils). In some embodiments, the pitch angle of spring (D) is approximately 11° (e.g., 10°, 12°, or 13°). In some embodiments, the spring constant (K) is approximately 0.5 lb./in to approximately 10 lb./in (e.g., 1 lb./in-5 lb./in, 3 lb./in-7 lb./in, 5 lb./in-10 lb./in). In some embodiments, (RFS) comprises a tandem foot allowing interconnectivity between the bone flap and native bone at time of placement and rotation. In some embodiments, (RFS) comprises a single foot allowing the device to be temporarily attached to the bone flap followed by placement of the flap and using rotating base (SB) to align and fixate the flap to native bone (see, e.g., FIG. 32). In some embodiments, a portion of base (SB) protrudes from the profile of the flap and native bone to allow for manual access, e.g., to allow for manual adjustment of the device utilizing thumb handle (H). In some embodiments, wherein base (SB) protrudes from the profile of the flap and native bone, (RFS) is able to be manually placed, adjusted, and/or removed as needed without the need for use of a second tool, e.g., a screwdriver or mallet). In some embodiments base (SB) has a height of approximately 4 mm to approximately 20 mm (e.g., 4 mm-10 mm, 7 mm-15 mm, 9 mm-20 mm). In some embodiments, base (SB) has an outer diameter of approximately 5 mm to approximately 30 mm (e.g., 5 mm-15 mm, 10 mm-20 mm, 12 mm-25 mm, 20 mm-30 mm). In some embodiments, base (SB) comprises an inner diameter of approximately 1 mm to approximately 10 mm (e.g., 1 mm-3 mm, 2 mm-7 mm, 5 mm-10 mm). In some embodiments, base (SB) has an indentation width of approximately 1 mm to approximately 10 mm (e.g., 1 mm-5 mm, 3 mm-7 mm, 4 mm-9 mm). In some embodiments, (RFS) may comprise a foot (F) that can be extended from a retracted state (FR) to an extended state (FE) by compression of the spring (D) to allow placement and or alignment for flaps ranging from 1 mm-20 mm (e.g., 5 mm-15 mm, 2 mm-10 mm, 10 mm-20 mm), (see, e.g., FIG. 30). In some embodiments, foot (F) is curved at an angle of approximately 90°, ± 7° (e.g., ± 5°, ± 12°, ±20°). In some embodiments, foot (F), after the curve, has a length of approximately 1 mm to approximately 10 mm (e.g., 1 mm-5 mm, 3 mm-7 mm, 4 mm-10 mm). In some embodiments, the diameter of the foot is less than or equal to the thickness of the kerf, e.g., 0.5 mm-5 mm (e.g., 1 mm-3 mm, 2 mm-5 mm). In some embodiments, foot (F) has a total length, not including the portion of the foot past the curve, of approximately 15 mm to approximately 45 mm (e.g., 15 mm-20 mm, 20 mm-30 mm, 25 mm-40 mm, 35 mm-45 mm). In some embodiments, foot (F) exerts force only on the posterior surface of the flap, e.g., force upward versus gravity, rather than exerting any force downward onto the anterior surface of the flap, e.g., force downward with gravity. In some embodiments, the foot assembly only engages the flap, e.g., it does not exert any force on the native bone. In some embodiments, several such (RFS)s are spaced out over the run of the osteotomy, or other injury site and kerf space to provide uniform spacing for placement of an adhesive composition. In some embodiments, said adhesive composition is comprised of a biomaterial or a combination of biomaterials. In some embodiments, said adhesive composition is comprised of a resorbable bone substitute. In some embodiments, some components of (RFS), e.g., foot (F) or base (SB), are comprised of an adhesive composition, e.g., a resorbable biocompatible material that disperses, e.g., a bone substitute. In some embodiments, the resorbable biocompatible material is replaced with host tissue over time. In some embodiments, said RFSs may be comprised of a compressible material to allow manual adjustment of the flap alignment into position.

In some embodiments, (RFS)s are of sufficient size and strength to withstand placement, adjustment, and removal without compromising the flap’s position. In some embodiments (RFS)s may remain in the kerf upon completion of the flap fixation procedure. In some embodiments (RFS)s are used intraoperatively to allow for temporary fixation during administration of an adhesive composition. In some embodiments (RFS)s can be removed intraoperatively once the adhesive composition reaches sufficient strength or rigidity to maintain bone flap fixation on a standalone basis. In some embodiments said (RFS)s remain in the kerf during injection and setting of the adhesive composition. In some embodiments, once each device has been removed, the adhesive composition is delivered to the gaps left remaining where the devices had been. In some embodiments, placement of the adhesive composition provides for a complete seal around the kerf space. In certain embodiments, (RFS)s may remain in the kerf during the placement of another device, e.g., another device described herein. In some embodiments, (RFS) weighs between about 1 g and 1000 g, depending on the material composition and size of the device.

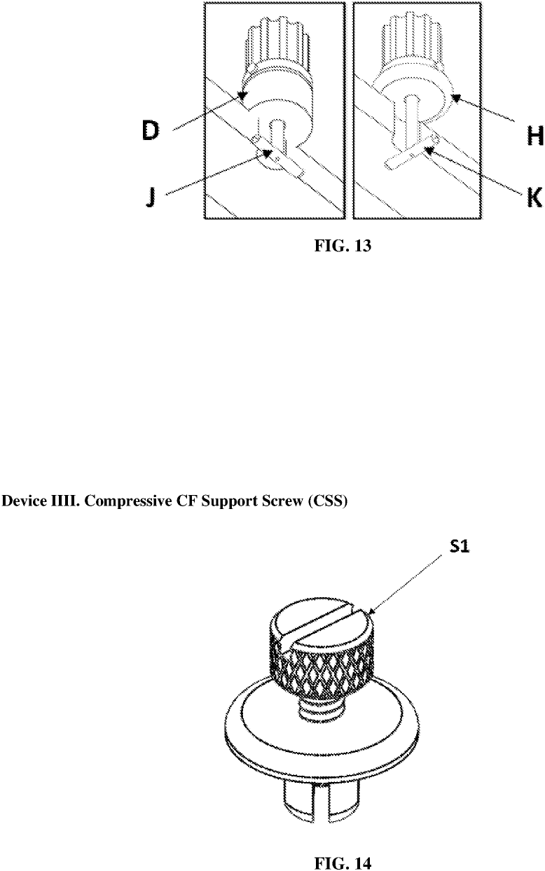

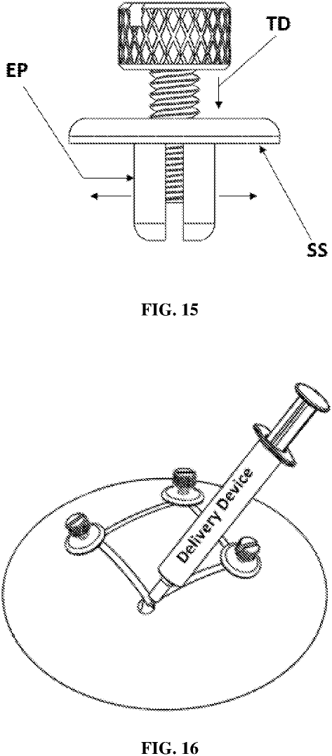

In another embodiment, the present disclosure features a compressive flap support screw device (CSS). In some embodiments, (CSS) comprises one or more of: a toggle assembly comprising a plastic base and upper support; a threaded screw (S1); and toggle expanding protrusions (EP). The toggle assembly may comprise a base (e.g., a plastic base) with a support. In some embodiments, screw (S1) is rotated, e.g., resulting in expansion of the toggle assembly such that the toggle expanding protrusions (EP) expand. Screw (S1) may be a threaded screw. In some embodiments, expanding protrusions (EP) expand to provide a force to fixate the flap and provide alignment of the flap to allow for delivery of an adhesive composition (FIGS. 14-15). In some embodiments, the width of the upper support (SS) is sufficient to span the kerf space width. In some embodiments, alignment and fixation of the flap utilizing one or more (CSS) allows for the delivery of an adhesive composition (FIG. 16). In some embodiments, said upper support (SS) comprises pre-drilled transition holes having sufficient contact with the native site. For example, the transition holes in the (SS) may contact both the native bone and flap wherein once threaded, (SS) will retain the flap in position in contact with the native bone. In some embodiments, the contact surface of the upper support (SS) has protrusions which can be fitted to accommodate the kerf space width, allowing the kerf width to be consistent along the entire kerf line. In some embodiments, said device is removable and adjustable, e.g., to allow manual adjustment of the flap if needed. In some embodiments, (CSS) further comprises a threaded thumb screw (TD). In some embodiments, the depths of said expandable protrusions (EP) are of sufficient length to provide sufficient force to retain the flap in position. In some embodiments, the protrusions (EP) do not protrude past the kerf thickness. In some embodiments, the toggle kerf contact surface may be flat or contoured to avoid sharp edges. In some embodiments, multiple devices may be placed around the kerf space as needed (FIG. 16). In some embodiments, (CSS) exerts force only on the posterior surface of the flap, e.g., force upward versus gravity, rather than exerting any force downward onto the anterior surface of the flap, e.g., force downward with gravity, e.g., wherein a craniotomy has been performed, force downward onto the skull. In some embodiments, (CSS) only engages the flap, e.g., it does not exert any force on the native bone.

In some embodiments, several such (CSS) devices may be distributed into the kerf space to provide uniform spacing for delivery of an adhesive composition. In some embodiments, said adhesive composition comprises a biomaterial or a combination of biomaterials. In some embodiments, said biomaterial comprises a bone substitute. In some embodiments, components of (CSS), e.g., thumb screw (TD), comprises a resorbable biocompatible material that disperses, e.g., a bone substitute. In some embodiments, said resorbable biocompatible material is replaced with host tissue over time. In some embodiments, the (CSS) comprises a compressible material to allow manual adjustment of the flap alignment into position. In some embodiments, said device is of sufficient size or strength to withstand placement, adjustment, and removal without compromising the flap’s position. In some embodiments said (CSS) remains in the kerf space upon completion of the flap fixation procedure. In some embodiments said (CSS) are used intraoperatively to allow for temporary fixation during administration of an adhesive composition. In some embodiments, a portion of (CSS) protrudes from the profile of the flap and native bone to allow for manual access, e.g., to allow for manual adjustment of the device utilizing thumb screw (TD). In some embodiments, wherein a portion of (CSS) protrudes from the profile of the flap and native bone, (CSS) is able to be manually placed, adjusted, and/or removed as needed without the need for use of a second tool, e.g., a screwdriver or mallet. In some embodiments said (CSS)s are removed intraoperatively once the adhesive composition reaches sufficient strength or rigidity to maintain bone flap fixation. In some embodiments said devices remain in the kerf space during injection and setting of the adhesive composition. In some embodiments, once each device has been removed, the adhesive composition is delivered to the gaps left remaining where the devices had been. In some embodiments, this provides for a complete seal around the kerf space. In certain embodiments, (CSS) remains in the kerf space during the placement of another device, e.g., another device described herein. In some embodiments, (CSS) weighs between about 1 g and 1000 g, depending on the material composition and size of the device.

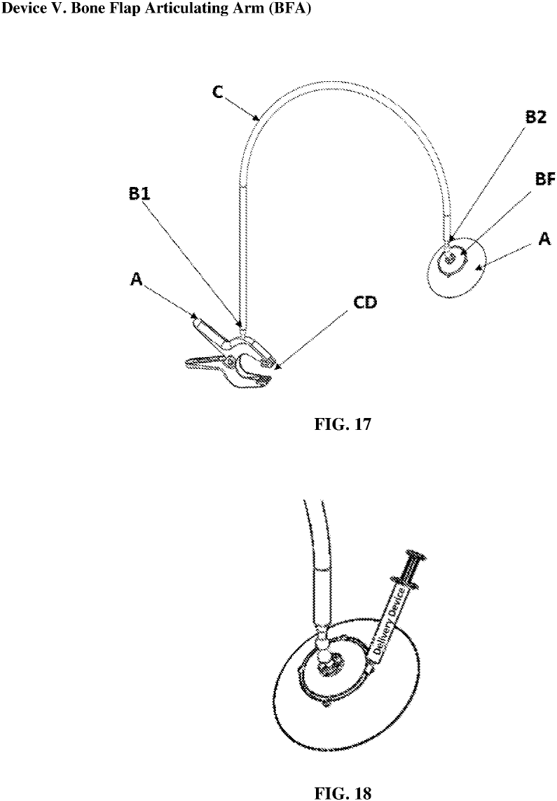

In another aspect, the present disclosure features a bone flap articulating device (BFA). In some embodiments, the device is used to temporarily fixate a flap in place using an attached articulating and position lockable arm. In some embodiments, arm (A) of (BFA) may be lockable. In some embodiments, said arm (A) is temporarily anchored to a surgical working surface (FIG. 17). In some embodiments, said arm (A) is part of a surgical robot. In some embodiments, once affixed or in position, arm (A) can be manipulated for surgical field use. In some embodiments, an adapter is temporarily attached to the flap at the distal end of (BFA) using screws, protrusions, hook and loop, vacuum cup, or temporary gel adhesive, or some other such attachment method. In some embodiments, arm (A) is of such a design that allows for semi-rigid adjustment with the ability to manipulate the flap into alignment with its position (FIG. 18). In some embodiments, (BFA) exerts force only on the posterior surface of the flap, e.g., force upward versus gravity, rather than exerting any force downward onto the anterior surface of the flap, e.g., force downward with gravity, e.g., wherein a craniotomy has been performed, force downward onto the skull. In some embodiments, (BFA) only engages the flap, e.g., it does not exert any force on the native bone. In some embodiments, one or more (BFA)s may be attached to the flap to allow it to be fixated position thus providing uniform spacing for placement of an adhesive composition. In some embodiments, said adhesive composition is comprised of a biomaterial or a combination of biomaterials. In some embodiments, said biomaterial is comprised of a bone substitute. In some embodiments, components of (BFA), e.g., arm (A), comprises a resorbable biocompatible material that disperses, e.g., a bone substitute. In some embodiments, said resorbable biocompatible material is replaced with host tissue over time. In some embodiments, the (BFA) comprises a compressible material to allow manual adjustment of the flap alignment into its original or corrected position. In some embodiments, said (BFA) is of sufficient size and strength to withstand placement, adjustment, and removal without compromising the flap’s position. In some embodiments said (BFA)s are used intraoperatively to allow for temporary fixation during administration of an adhesive composition. In some embodiments, a portion of (BFA) protrudes from the profile of the flap and native bone to allow for manual access, e.g., to allow for manual adjustment of the device utilizing arm (A). In some embodiments, wherein a portion of (BFA) protrudes from the profile of the flap and native bone, (BFA) is able to be manually placed, adjusted, and/or removed as needed without the need for use of a second tool, e.g., a screwdriver or mallet. In some embodiments said (BFA)s are removed intraoperatively, e.g., once the adhesive composition reaches sufficient strength or rigidity to maintain bone flap fixation on a standalone basis. In some embodiments, part(s) of said device remain in the kerf space during injection and setting of the adhesive composition. In some embodiments, once each device has been removed, the adhesive composition is delivered to the gaps left remaining where the devices had been. In some embodiments, this provides for a complete seal around the kerf space. In certain embodiments, (BFA)s remain in the kerf space during the placement of another device, e.g., another device described herein. In some embodiments, (BFA) weighs between about 10 g and 10,000 g, depending on the material composition and size of the device.

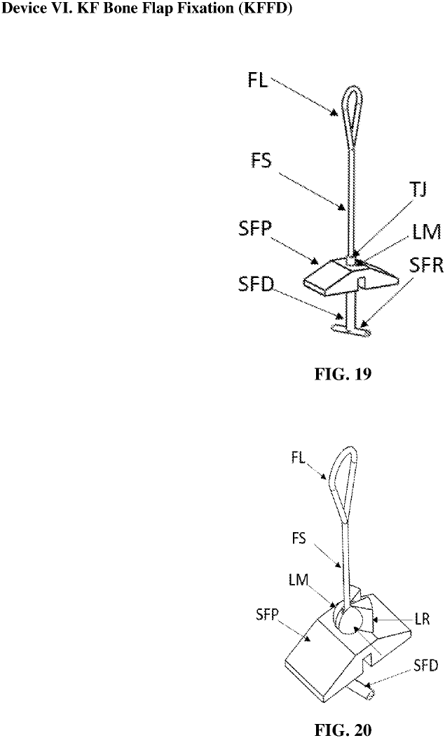

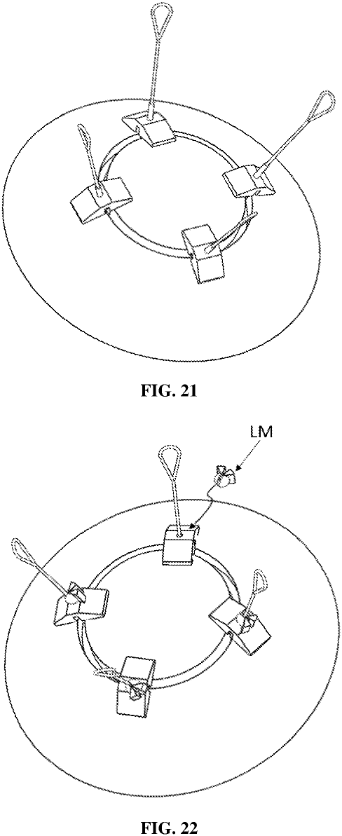

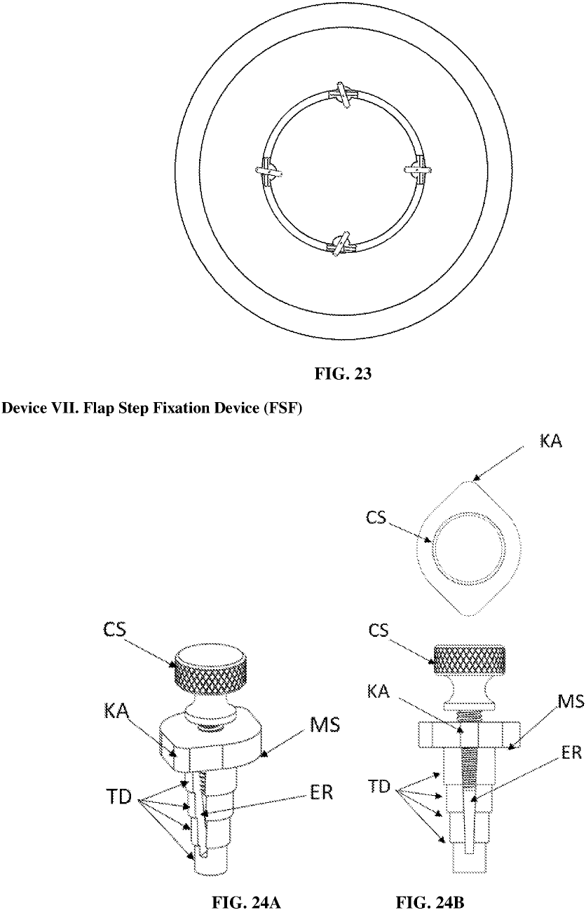

In another aspect, the present disclosure features a kerf flap fixation device (KFFD). Said (KFFD) may comprise one or more of: a support foot distal (SFD); a support foot proximal (SFP); a flexible proximal loop (FP); a flexible shaft (FS); and a locking mechanism (LM) (FIGS. 19-20). In some embodiments, support foot distal (SFD) is placed into the kerf using a transition hole created by a drill or bur (FIGS. 19-20). In some embodiments, (KFFD) may be rotated to where support foot distal (SFD) is 30-90° opposed to the kerf line (FIG. 20). In some embodiments, support foot proximal (SFP) may be fitted over the flexible proximal loop (FP) and flexible shaft (FS), which is attached to the support foot distal (SFD). In some embodiments, upon placement, the user provides a gentle tensile force onto the support foot distal (SFD) while pressing support foot proximal (SFP) onto the native bone surface (FIGS. 21-23). In some embodiments, once (SFP) is introduced to support foot distal (SFD), a locking feature (LM) is engaged. In some embodiments, locking mechanism (LM) comprises friction, compressed spring (linear or coil) or other mechanical means that once engaged provides support to fixate the flap in position (FIG. 22). In some embodiments, flexible proximal loop (FP) may be a braided or bonded strand or weave of multiple strands creating a loop of about 5 mm to about 50 mm (e.g., 5 mm to 25 mm, 10 mm to 40 mm, 25 mm to 50 mm, 5 mm to 25 mm, or 20 mm to 40 mm) in diameter. In some embodiments, (KFFD) is of a design that allows for semi-rigid adjustment to allow a user to manipulate the bone flap into its position. In some embodiments, upon completion of bone flap fixation, the locking mechanism is released, thereby allowing the device to be separated from the native bone and bone flap and removed. In some embodiments, support foot distal (SFD) may have a transition joint (TJ) and shaft diameter of about 0.018″to about 0.250″. In some embodiments, (SFD) may be of a length from about 3 mm to about 25 mm. In some embodiments, the (SFD) overall length prior to (FP) and (FS) is between about 5 mm to about 50 mm. In some embodiments, flexible loop (FL) and flexible shaft (FS) are made of a single or braided metallic material, or polymer strand or braided extrusion. In some embodiments, the transition joint (TJ) is made using welding, solvent bonding, RF welding, knotted slide assembly, or other similar method. In some embodiments, flexible shaft (FS) may be about 1 cm to about 100 cm (e.g., 1 cm to 25 cm, 1 cm to 50 cm, 25 cm to 100 cm, 50 cm to 100 cm, 75 cm to 100 cm, 50 cm to 75 cm, or 50 cm to 100 cm) in length. In some embodiments, support foot proximal (SFP) may have a width of about 3 mm to about 20 mm (e.g., 3 mm to 10 mm, 5 mm to 10 mm, 10 mm to 15 mm, 10 mm to 20 mm). In some embodiments, support foot proximal (SFP) may have an overall length of approximately 10 mm to about 50 mm (e.g., 10 mm to 25 mm, 25 mm to 50 mm). Support foot proximal (SFP) may have a central relief of about 0.02″ to about 0.25″ (e.g., 0.02″ to 0.1″, 0.5 to 0.1″, or 1″ to 0.25″).

In some embodiments (SFP) has a locking mechanism (LM) that is friction based on the outer diameter of support foot distal (SFD) and inner diameter of support foot distal (SFD). Optionally, locking mechanism (LM) may be created by a ratcheting mechanism. In some embodiments, (SFP) has a locking mechanism (LM) that is created using a compressive load provided by a captured coil or leaf spring. In some embodiments, locking mechanism (LM) is overcome by releasing a button that compresses the spring or other compressive load. In some embodiments, support foot proximal (SFP) has a pre-created relief that can be broken by the user using surgical tools for removal. In some embodiments, (SFP) is affixed to the bone surface using a removable adhesive, vacuum cups, magnetic, pneumatic bladders, hook and loop materials, or other similar such fixation methods. In some embodiments, locking mechanism (LM) may be established using a separate device, e.g., lock receptor (LR) designed to capture flexible shaft (FS) by compressing the outer surface of (LM) while maintaining tangent contact with the proximal surface of support foot proximal (SFP) (FIG. 22). In some embodiments, locking mechanism (LM) may be released by compressing lock receptor (LR), thus separating the device and releasing (FS). In some embodiments, locking mechanism (LM) maybe comprised of a separate device to temporarily secure (SFD) and (SFP) until removal is needed. In some embodiments, (KFFD) exerts force only on the posterior surface of the flap, e.g., force upward versus gravity, rather than exerting any force downward onto the anterior surface of the flap, e.g., force downward with gravity, e.g., wherein a craniotomy has been performed, force downward onto the skull. In some embodiments, (KFFD) only engages the flap, e.g., it does not exert any force on the native bone. In some embodiments, several such (KFFD)s may be distributed into the kerf space to provide uniform spacing for delivery of an adhesive composition. In some embodiments, said adhesive composition is comprised of a biomaterial or a combination of biomaterials. In some embodiments, said biomaterial comprises a bone substitute. In some embodiments, components of (KFFD), e.g., support foot distal (SFD), are comprised of a resorbable biocompatible material that disperses, e.g., a bone substitute. In some embodiments the resorbable biocompatible material is replaced with host tissue over time. In some embodiments, said (KFFD)s are comprised of compressible and sturdy materials to allow for manual alignment of the flap into position. In some embodiments, said (KFFD)s are of sufficient size or strength to withstand placement, adjustment, and removal without compromising the flap’s position. In some embodiments said (KFFD)s remain in the kerf space upon completion of the flap fixation procedure. In some embodiments, (KFFD)s are used intraoperatively to allow for temporary fixation during delivery of an adhesive composition. In some embodiments, a portion of (KFFD) protrudes from the profile of the flap and native bone to allow for manual access, e.g., to allow for manual adjustment of the device. In some embodiments, wherein a portion of (KFFD) protrudes from the profile of the flap and native bone, (KFFD) is able to be manually placed, adjusted, and/or removed as needed without the need for use of a second tool, e.g., a screwdriver or mallet. In some embodiments, (KFFD)s are removed intraoperatively, e.g., once the adhesive composition reaches sufficient strength or rigidity to maintain bone flap fixation on a standalone basis. In some embodiments, said (KFFD)s remain in the kerf space during delivery and setting of the adhesive composition. In some embodiments, once each device has been removed, the adhesive composition is delivered to the gaps left remaining where the devices had been. In some embodiments, this provides for a complete seal around the kerf space. In some embodiments, said (KFFD)s remain in the kerf space during the placement of another device, e.g., another device described herein. In some embodiments, (KFFD)s weigh between about 1 g and 1000 g, depending on the material composition and size of the device.

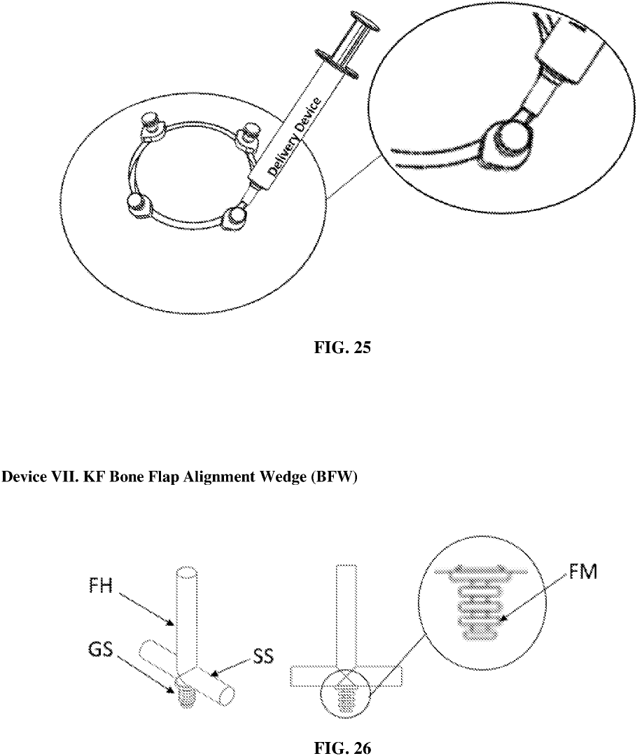

In another aspect, the present disclosure features a flap step fixation device (FSF) comprising one or more of: an alignment feature (KA); an outer wall (TD); and a compression screw (CS). In some embodiments, said alignment feature (KA) utilizes a compression screw (CS) to stress the outer wall (TD) until it expands, providing support for temporary fixation of the flap in position (FIGS. 24A-B). In some embodiments, (FSF) provisionally fixates the flap by placing a plurality of said (FSF)s into pre-drilled kerf transition hole(s). Said (FSF)s may be aligned such that they are parallel to the kerf space using alignment feature (KA). In some embodiments, compression screw (CS) may then be placed in the center of alignment feature (KA) and rotated in order to stress the outer wall (TD) such that the device expands, providing support along the kerf transition hole’s inner diameter. In some embodiments, a plurality of (FSF)s hold the flap in its position for the delivery of an adhesive composition (FIG. 25). In some embodiments, (FSF) exerts force only on the posterior surface of the flap, e.g., force upward versus gravity, rather than exerting any force downward onto the anterior surface of the flap, e.g., force downward with gravity, e.g., wherein a craniotomy has been performed, force downward onto the skull. In some embodiments, (FSF) only engages the flap, e.g., it does not exert any force on the native bone. In some embodiments, several such (FSF)s may be distributed into the kerf space to provide uniform spacing for delivery of an adhesive composition. In some embodiments, said adhesive composition is comprised of a biomaterial or a combination of biomaterials. In some embodiments, components of (FSF), e.g., compression screw (CS), are comprised of a resorbable biocompatible material that disperses, e.g., a bone substitute. In some embodiments, the resorbable biocompatible material is replaced with host tissue over time. In some embodiments, said (FSF)s are comprised of a malleable material to allow for manual alignment of the flap into its position. In some embodiments, said devices are of sufficient size or strength to withstand placement, adjustment, and removal without compromising the flap’s position. In some embodiments said (FSF)s remain in the kerf space upon completion of the flap fixation procedure. In some embodiments, said (FSF)s are used intraoperatively, e.g., to allow for temporary fixation during delivery of an adhesive composition. In some embodiments, a portion of (FSF) protrudes from the profile of the flap and native bone to allow for manual access, e.g., to allow for manual adjustment of the device utilizing compression screw (CS). In some embodiments, wherein a portion of (FSF) protrudes from the profile of the flap and native bone, (FSF) is able to be manually placed, adjusted, and/or removed as needed without the need for use of a second tool, e.g., a screwdriver or mallet. In some embodiments, said (FSF)s are removed intraoperatively once the adhesive composition reaches sufficient strength or rigidity to maintain bone flap fixation on a standalone basis. In some embodiments, (FSF)s remain in the kerf space during delivery and setting of the adhesive composition. In some embodiments, once each device has been removed, the adhesive composition is delivered to the gaps left remaining where the devices had been. In some embodiments, this provides for a complete seal around the kerf space. In some embodiments, (FSF)s remain in the kerf space during the placement of another device, e.g., another device described herein. In some embodiments, an exemplary (FSF) weighs between about 1 g and 1000 g, depending on the material composition and size of the device.

In another aspect, the present disclosure features a bone flap alignment wedge device (BFW) which may comprise one or more of: a grabbing surface (GS); a finger handle (FH); and support surface (SS). In some embodiments, said device may utilize finger handle (FH) to allow a user to place grabbing surface (GS) into the kerf line until sealed. In some embodiments, grabbing surface (GS) may comprise a soft durometer material to maintain fixation in the kerf (FIG. 26). In some embodiments, (BFW) features support surface (SS) to align the flap parallel with the native bone (FIG. 28). In some embodiments, said (BFW) is removed by gentle rotation and tension applied by the user to fully retract (BFW) from the kerf space. In some embodiments, (BFW) is comprised of a biocompatible material. Said biocompatible material comprises organic or synthetic matter, or a combination of both. In some embodiments, said (BFW) is comprised of a combination of biocompatible materials to allow better rigidity of support surface (SS) and finger handle (FH) when being placed, and such that grabbing surface (GS) may be flexible in aligning and holding the flap in its position. In some embodiments, grabbing surface (GS) may be comprised of butyl rubber, silicone, or other such malleable biocompatible materials. In some embodiments, grabbing surface (GS) is attached to finger handle (FH) via compression forces or adhesive materials, or via welding, brazing or soldering. In some embodiments, grabbing surface (GS) and finger handle (FH) may be comprised of one piece. In some embodiments, support surface (SS) may be of a circular shape or a bar. In some embodiments, support surface (SS) comprises translucent materials to allow for better view of the placement site and alignment. In some embodiments, (BFW) exerts force only on the posterior surface of the flap, e.g., force upward versus gravity, rather than exerting any force downward onto the anterior surface of the flap, e.g., force downward with gravity, e.g., wherein a craniotomy has been performed, force downward onto the skull. In some embodiments, (BFW) only engages the flap, e.g., it does not exert any force on the native bone. In some embodiments, several such (BFW)s may be distributed into the kerf space to provide uniform spacing for delivery of an adhesive composition. In some embodiments, said adhesive composition comprises a biomaterial or a combination of biomaterials. In some embodiments, components of (BFW), e.g., grabbing surface (GS) may be comprised of a resorbable biocompatible material that disperses, e.g., a bone substitute. In some embodiments, said resorbable biocompatible material is replaced by host tissue over time.

In some embodiments, said (BFW)s are comprised of a malleable material to allow manual alignment of the flap into position. In some embodiments, said (BFW)s are of sufficient size or strength to withstand placement, adjustment, and removal without compromising the flap’s position. In some embodiments, said (BFW)s remain in the kerf upon completion of the flap fixation procedure. In some embodiments, said (BFW)s are used intraoperatively to allow for temporary fixation during delivery of an adhesive composition. In some embodiments, a portion of (BFW) protrudes from the profile of the flap and native bone to allow for manual access, e.g., to allow for manual adjustment of the device utilizing finger handle (FH). In some embodiments, wherein (FH) protrudes from the profile of the flap and native bone (BFW) is able to be manually placed, adjusted, and/or removed as needed without the need for use of a second tool, e.g., a screwdriver or mallet. In some embodiments, said (BFW)s are removed intraoperatively, e.g., once the adhesive composition reaches sufficient strength or rigidity to maintain bone flap fixation on a standalone basis. In some embodiments, said (BFW)s remain in the kerf space during delivery and setting of the adhesive composition. In some embodiments, once each device has been removed, the adhesive composition is delivered to the gaps left remaining where the devices had been. In some embodiments, this provides for a complete seal around the kerf space. In some embodiments, said (BFW)s remain in the kerf space during the placement of another device, e.g., another device described herein. In some embodiments, (BFW) weighs between about 1 g and 1000 g, depending on the material composition and size of the device.

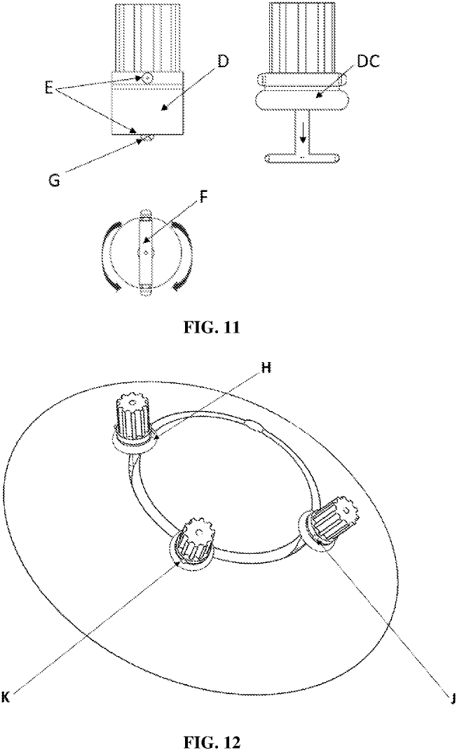

In another aspect, the present disclosure features a further embodiment, wherein a plurality of cranial flap fixation support (CFS)s may be distributed spaced out over the run of the osteotomy, e.g., a craniotomy, created by a surgeon. In some embodiments, once the flap is placed into its desired location next to the native bone, said (CFS)s are inserted into the kerf in previously determined spots so as to be evenly spaced about the kerf line for an even hold. In some embodiments, foot (F) of the device is aligned with the center of the kerf width to provide a roughly uniform separation between the edges of the flap to be affixed, e.g., a cranial flap, and the cut edge of the native bone. In some embodiments, once the flap is placed into position, a series of (CFS)s are inserted into relevant positioning by rotating the rotating handle (A) which acts to extend the foot (F) from its closed position to an open and extended position (FIG. 11). In some embodiments, once foot (F) has been extended, it is placed into the kerf line with the foot (F) parallel to the kerf line. Once (CFS) has been placed into the kerf line, rotating handle (A) may be turned in the opposite direction so as to rotate foot (F) to be perpendicular to the kerf line. In some embodiments, such orientation fixates the bone flap against the native bone via the force created on the flap and native bone by being compressed between either side of foot (F) and the base (B) of the device. In some embodiments, rotating handle (A) is rotated enough that foot (F) is pulled tightly against the bone flap to create a stronger force on the bone flap between foot (F) and base (B). In some embodiments, (CFS) exerts force only on the posterior surface of the flap, e.g., force upward versus gravity, rather than exerting any force downward onto the anterior surface of the flap, e.g., force downward with gravity, e.g., wherein a craniotomy has been performed, force downward onto the skull. In some embodiments, (CFS) only engages the flap, e.g., it does not exert any force on the native bone. In some embodiments, (CFS) also features a compression washer (DC) which acts to provide some cushion and protection for the bone flap or native bone against the rest of the (CFS). In some embodiments, rotating handle (A) features indents for easier handling. In some embodiments, (CFS) features a knob (C) on the front of rotating handle (A) for fine-tuned handling as necessary. In some embodiments, a plurality of (CFS)s are spaced out evenly throughout the kerf space to provide an even hold (FIG. 12). In some embodiments, once alignment and fixation have been achieved, an adhesive composition is delivered. In some embodiments, a portion of (CFS) protrudes from the profile of the flap and native bone to allow for manual access, e.g., to allow for manual adjustment of the device utilizing knob (C). In some embodiments, wherein a portion of (CFS) protrudes from the profile of the flap and native bone, (CFS) is able to be manually placed, adjusted, and/or removed as needed without the need for use of a second tool, e.g., a screwdriver or mallet. In some embodiments, once the adhesive composition has set, each (CFS) is removed by rotating handle (A) to align foot (F) parallel to the kerf line and carefully removing the device from the kerf space. Once each device has been removed, the adhesive composition may be delivered to the gaps left remaining where the devices had been. In some embodiments, this provides for a complete seal around the kerf space. In some embodiments, said (CFS)s remain in the kerf space during the placement of another device, e.g., another device described herein. In some embodiments, (CFS) weighs between about 1 g and 1000 g, depending on the material composition and size of the device.

In another aspect, the present disclosure features a further embodiment, wherein a plurality of cranial flap fixation supports (CFFS)s may be distributed spaced out over the run of the osteotomy, e.g., a craniotomy, created by a surgeon. In some embodiments, once the flap is placed into its desired location next to the native bone, said (CFFS)s are inserted into the kerf in previously determined spots so as to be evenly spaced about the kerf line for an even hold. In some embodiments, each (CFFS) device comprises a base (B), a handle (H), a top clamp (TC), and a bottom clamp (BC) (FIG. 33). In some embodiments, top clamp (TC) has a curved, blunt end with no clamping teeth or bit so as to hold the flap or native bone via a compressive force only. In some embodiments, bottom clamp (BC) comprises a single, soft-edged clamping bit, which acts to hold the bone flap in place from the underside. In some embodiments, a (CFFS) is placed into the kerf space by: holding base (B) in one hand and turning handle (H) such that bottom clamp (BC) is in an open position; sliding bottom clamp (BC) on the underside of the bone flap with the top clamp (TC) holding the top of the bone flap firmly; turning handle (H) in the opposite direction as previously so that bottom clamp (BC) tightens its grip on the underside of the bone flap, such that the flap becomes held firmly in place between bottom clamp (BC) and top clamp (TC). In some embodiments, base (B) acts to ensure the bone flap does not move relative to the native bone to which it is being fixated. In some embodiments, a plurality of (CFFS)s are spaced out evenly throughout the kerf space to provide an even hold. In some embodiments, (CFFS) exerts force only on the posterior surface of the flap, e.g., force upward versus gravity, rather than exerting any force downward onto the anterior surface of the flap, e.g., force downward with gravity, e.g., wherein a craniotomy has been performed, force downward onto the skull. In some embodiments, (CFFS) only engages the flap, e.g., it does not exert any force on the native bone. In some embodiments, once alignment and fixation have been achieved, an adhesive composition is delivered to the kerf space. In some embodiments, a portion of (CFFS) protrudes from the profile of the flap and native bone to allow for manual access, e.g., to allow for manual adjustment. In some embodiments, wherein a portion of (CFFS) protrudes from the profile of the flap and native bone, (CFFS) is able to be manually placed, adjusted, and/or removed as needed without the need for use of a second tool, e.g., a screwdriver or mallet. In some embodiments, once the adhesive composition has set, each (CFFS) is removed by rotating handle (H) to loosen bottom clamp (BC), at which point each (CFFS) can be carefully taken out of the kerf space. Once each device has been removed, the adhesive composition may be delivered to the gaps left remaining where the devices had been. In some embodiments, this provides for a complete seal around the kerf space.

The flap fixation devices described herein may engage with the bone in a variety of ways. For example, an exemplary device may grip the bone surface, e.g., via spring force, or an exemplary device may pierce the bone, e.g., by implanting into the bone. In some embodiments, the flap fixation device grips the bone surface and applies a force to, e.g., join the cranial flap to the cranium. In some embodiments, the device grips the bone surface without entering the bone, e.g., without breaking the bone surface or without causing injury to the bone. In some embodiments, the device acts as a wedge between the native bone and the flap being fixated, in order to temporarily fixated the flap in that location. Exemplary flap fixation devices that engage with the bone by gripping the surface are described herein, such as (RFS), (BFW), (KFFD) and (CFS).

In other embodiments, an exemplary device engages with and fixates the bone flap by partially or fully implanting the exemplary flap fixation device into the bone. For example, an exemplary flap fixation device may screw into the bone surface temporarily fixate the flap in position. In some embodiments, an exemplary flap fixation device utilizes a screw force and compression force against the walls of the kerf space to fixate the flap in position. In some embodiments, the screw portion of the exemplary flap fixation device only enters the bone of the flap to be fixated. In other embodiments, the screw portion of the exemplary flap fixation device enters the bone on the bone flap and the native bone. In some embodiments, exemplary flap fixation devices utilizing screw fixation do not enter the bone past the point necessary to fixate the flap. In some embodiments, once an adhesive composition has been delivered to the kerf space, the exemplary fixation devices can be removed and the space left behind filled with adhesive composition, wherein the screw holes will be filled in and the adhesive composition will be resorbed and replaced with native bone. Exemplary flap fixation devices that engage with the bone by implanting into the bone surface are described herein, such as (CFFR), (CFR), and (CSS).

The devices described herein may engage with the bone flap to be fixated at one position along the cranial flap or at a plurality of positions along the cranial flap. For example, an exemplary flap fixation device may comprise one position for gripping the bone for temporary fixation. Exemplary flap fixation devices, as described herein, which grip the surface, without implanting into the bone, at one point of contact include (BFW), (KFFD), and (RFS). In some embodiments, the single -point bone flap fixation devices require contact with the bone at only one position in order to achieve the goal of temporary bone flap fixation to aid in the delivery of an adhesive composition. In some embodiments, said single-point bone flap fixation devices minimize points of surgical contact, thus minimizing risk of infection at the surgical site. In some embodiments, where fewer points of contact are needed to engage a flap fixation device with a bone flap to be fixated, fixation is achieved more efficiently.

In some embodiments, an exemplary flap fixation device which implants into the bone engages the bone at a single point of contact. Exemplary flap fixation devices, as described herein, which implant into the bone surface and engage the bone at a single point of contact include (CSS), (BFA), and (FSF). In some embodiments, the single-point bone flap fixation devices require contact with the bone at only one position in order to achieve the goal of temporary bone flap fixation to aid in the delivery of an adhesive composition. In some embodiments, said single-point bone flap fixation devices minimize points of surgical contact, thus minimizing risk of infection at the surgical site. In some embodiments, where fewer points of contact are needed to engage a flap fixation device with a bone flap to be fixated, fixation is achieved more efficiently.

In some embodiments, an exemplary flap fixation device engages the bone at a plurality of positions, e.g., two, three, four, or more positions. In an embodiment, an exemplary flap fixation device engages the bone without implanting into the bone at two positions along the bone surface, e.g., by gripping the bone or via compression or spring force, e.g., (KFFD). In some embodiments, an exemplary flap fixation device engages with the bone by implanting into the bone just deep enough to achieve temporary flap fixation, e.g., via screws or pegs. Exemplary devices that engage with the bone at two positions along the bone surface include, as described herein, include (CFFR), (CFS), and (KFFD). In some embodiments, the use of a plurality of flap fixation device engagement positions adds stability and strength to the flap fixation. In some embodiments, when a flap fixation device has a plurality of bone engagement positions, fewer devices may be needed overall to achieve the same level of fixation strength.

The above devices and embodiments may be used separately or in conjunction with each other. Features of one device may be seen in use or as part of any other device. For example, any of the devices may be used together to form an assembly of devices. In an embodiment, an assembly of devices comprises at least 2, e.g., 3, 4, 5, 6, 7, or 8 devices, each of which is the same style of device. In another embodiment, an assembly of devices comprises at least 2, e.g., 3, 4, 5, 6, 7, or 8 devices, one or more of which is a different style of device.

### Compositions

The present disclosure also features an adhesive composition for use with the device embodiments described herein. In some embodiments, the adhesive composition comprises a material that fills into the kerf space to partially or fully replace the bone removed from the operation site. In some embodiments, the adhesive composition is utilized to partially or fully seal the kerf line in the osteotomy. A partial fill refers to enabling permanent fixating of the flap, whereas a full fill refers to sealing the kerf space with a depth equaling the thickness of the flap and the circumference equaling the run of the osteotomy. In some embodiments, the adhesive composition comprises a biomaterial. Exemplary biomaterials may comprise carbon-based compounds, non-carbon-based compounds, or a combination thereof. In some embodiments, said biomaterial comprises one or more of: hydrogel (e.g., polyethylene glycol and propylene glycol, or pectin, carboxymethylcellulose and propylene glycol); cement (e.g., polymethyl methacrylate (PMMA) or calcium phosphate); or an adhesive (e.g., cyanoacrylates, polyurethanes, or fibrin glues). In some embodiments, said biomaterial comprises an adhesive composition. In some embodiments, said biomaterial comprises a resorbable material, e.g., a material which is resorbed by a subject and may be replaced with host tissue. In some embodiments, the biomaterial composition comprises a mineral-based compound comprising a multivalent metal, an organic compound and an aqueous medium. In some embodiments, said mineral-based compound may be a calcium, e.g., calcium phosphate. In some embodiments, the mineral-based compound comprises alpha tricalcium phosphate, beta tricalcium phosphate, or tetracalcium phosphate. In some embodiments, said organic-based compound may comprise an organophosphate or organic acid.

In some embodiments, the adhesive composition comprises a compound of Formula (I):

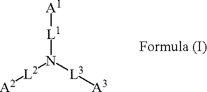

or a salt thereof, wherein: each of A¹, A², and A³ is independently selected from an acidic group (e.g., a carboxyl or phosphonyl); and each of L¹, L², and L³ is independently bond, alkylene (e.g., C₁-C₆ alkylene), or heteroalkylene (e.g., C₁-C₆ heteroalkylene).

In some embodiments, each of A¹, A², and A³ is independently a carboxyl or phosphonyl. In some embodiments, A¹ is carboxyl, and A² and A³ are phosphonyl. In some embodiments, A¹, A² and A³ are phosphonyl.

In some embodiments, each of L¹, L², and L³ is C₁-C₃ alkylene. In some embodiments, each of L¹, L², and L³ is C₁ alkylene.

In some embodiments, the compound of Formula (I) is a compound of Formula (I-a) or (I-b):

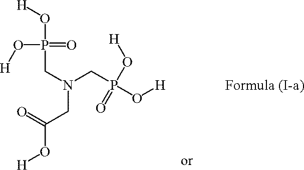

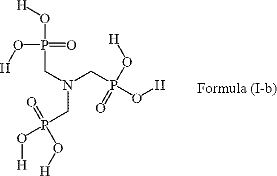 [SMILES: O=P(O)(O)CN(CP(=O)(O)O)CP(=O)(O)O]

In some embodiments, the adhesive composition comprises a compound of Formula (II):

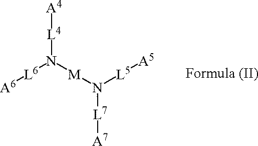

or a salt thereof, wherein: each of A⁴, A⁵, and A⁶, is independently selected from an acidic group (e.g., a carboxyl or phosphonyl); A⁷ is selected from an acidic group (e.g., a carboxyl or phosphonyl), a hydrogen atom, an alkyl, an aryl, a hydroxy group, a thio group, and an amino group; each of L⁴, L⁵, L⁶, and L⁷ is independently bond, alkylene (e.g., C₁-C₆ alkylene), or heteroalkylene (e.g., C₁-C₆ heteroalkylene); and M is alkylene (e.g., C₁-C₆ alkylene) or heteroalkylene (e.g., C₁-C₆ heteroalkylene).

In some embodiments, A⁴, A⁵, A⁶ and A⁷ are carboxyl.

In some embodiments, L⁴, L⁵, L⁶, and L⁷ are C₁-C₃ alkylene. In some embodiments, L⁴, L⁵, L⁶, and L⁷ are C₁ alkylene.

In some embodiments, M is C₁-C₄ alkylene. In some embodiments, M is C₂ alkylene. In some embodiments, M is C₃ alkylene. In some embodiments, M is C₁-C₆ heteroalkylene. In some embodiments, M is C₆ heteroalkylene. In some embodiments, M is bis(ethyleneoxy)ethylene. In some embodiments, M includes side chains. In some embodiments, M includes multiple side chains. In some embodiments, M includes one or multiple carboxymethylene side chains. In some embodiments, M includes one or multiple N-carboxymethylene groups or N-hydroxymethylene groups.

In some embodiments, the compound of Formula (II) includes three, four, five, six, or more N-carboxymethylene groups.

In some embodiments, the compound of Formula (II) comprises ethylenediamine tetraacetic acid (EDTA).

In some embodiments, the compound of Formula (II) is a compound of Formula (II-a), (II-b), (II-c), (II-d), (II-e), or (II-f):

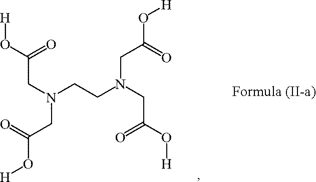

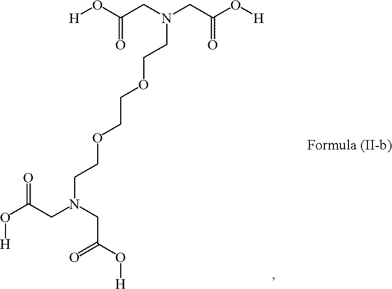

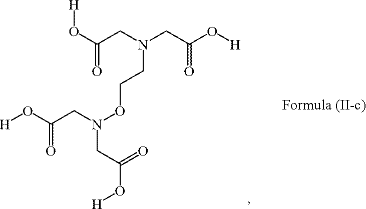

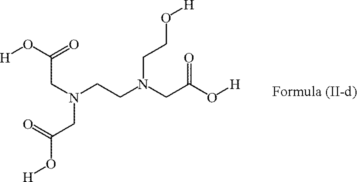 [SMILES: O=C(O)CN(CCO)CCN(CC(=O)O)CC(=O)O]

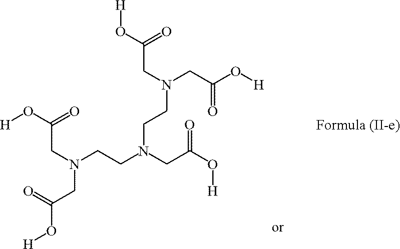

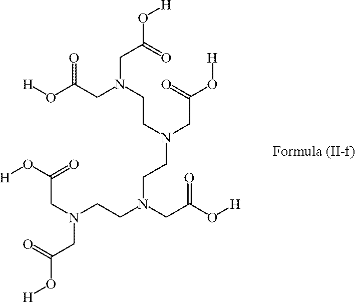 [SMILES: O=C(O)CN(CCN(CCN(CC(=O)O)CC(=O)O)CC(=O)O)CCN(CC(=O)O)CC(=O)O]

In some embodiments, the adhesive composition comprises a compound of Formula (III):

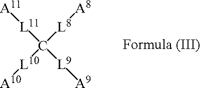

or a salt thereof, wherein: each of A⁸ and A⁹ is independently selected from an acidic group (e.g., a carboxyl or phosphonyl); each of A¹⁰ and A¹¹ is independently selected from an acidic group (e.g., a carboxyl or phosphonyl), a hydrogen atom, an alkyl, aryl, a hydroxy group, a thio group, and an amino group; each of L⁸, L⁹, L¹⁰ and L¹¹ is independently bond, alkylene (e.g., C₁-C₆ alkylene), or heteroalkylene (e.g., C₁-C₆ heteroalkylene).

In some embodiments, A⁸, A⁹, and A¹⁰ are carboxyl.

In some embodiments, A¹⁰, A¹¹, are a hydrogen atom.

In some embodiments, A¹¹ is a hydroxy or amino group.

In some embodiments, L⁸, L⁹, L¹⁰, and L¹¹ are a bond.

In some embodiments, L⁸ and L⁹ are C₁-C₃ alkylene.

In some embodiments L¹¹ is a heteroalkylene (e.g., C₁-C₆ heteroalkylene).

In some embodiments L¹¹ is methylenethiomethylene.

In some embodiments, the compound of Formula (III) comprises citric acid or malonic acid.

In some embodiments, the compound of Formula (III) is a compound of Formula (III-a), (III-b), (III-c), or (III-d):

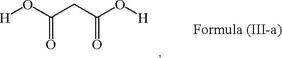

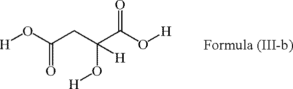

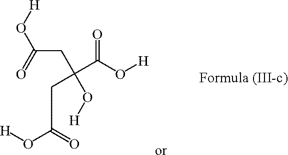

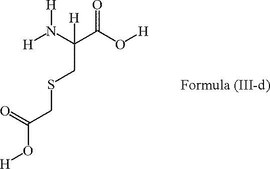 [SMILES: NC(CSCC(=O)O)C(=O)O]

In some embodiments, the adhesive composition comprises a compound of Formula (IV):

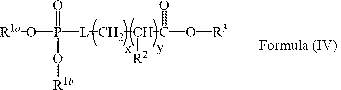

or a salt thereof, wherein: L is O, S, NH, or CH₂; each of R^{1a} and R^{1b} is independently H, an optionally substituted alkyl, or an optionally substituted aryl; R² is H, NR^{4a}R^{4b}, C(O)R⁵, or C(O)OR⁵; R³ is H, an optionally substituted alkyl, or an optionally substituted aryl; each of R^{4a} and R^{4b} is independently H, C(O)R⁶, or an optionally substituted alkyl; R⁵ is H, an optionally substituted alkyl, or an optionally substituted aryl; R⁶ is an optionally substituted alkyl or an optionally substituted aryl; and each of x and y is independently 0, 1, 2, or 3.

In some embodiments, L is O or S. In some embodiments, L is O. In some embodiments, each of R^{1a} and R^{1b} is independently H. In some embodiments, L is O, and each of R^{1a} and R^{1b} is H.

In some embodiments, R² is selected from H, NR^{4a}R^{4b}, and C(O)R⁵. In some embodiments, R² is NR^{4a}R^{4b}. In some embodiments, R² is NR^{4a}R^{4b} and each of R^{4a} and R^{4b} is independently H.

In some embodiments, L is O, each of R^{1a} and R^{1b} is independently H, R² is NR^{4a}R^{4b}, and each of R^{4a} and R^{4b} is independently H.

In some embodiments, R³ is H. In some embodiments, L is O, each of R^{1a} and R^{1b} is independently H, R² is NR^{4a}R^{4b}, each of R^{4a} and R^{4b} is independently H, and R³ is H.

In some embodiments, each of x and y is independently 0 or 1. In some embodiments, each of x and y is independently 1. In some embodiments, L is O, each of R^{1a} and R^{1b} is independently H, R² is NR^{4a}R^{4b}, each of R^{4a} and R^{4b} is independently H, R³ is H, and each of x and y is 1.

In some embodiments, the compound of Formula (IV) is phosphoserine.

In some embodiments, the adhesive composition comprises an organic compound of Formula (V):

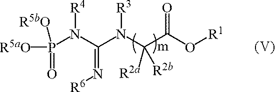

wherein R¹ is H, optionally substituted alkyl, optionally substituted alkyl, optionally substituted alkyl, optionally substituted aryl, or optionally substituted heteroaryl; each of R^{2a} and R^{2b} is independently H, optionally substituted alkyl, hydroxy, alkoxy, or halo; each of R³ and R⁴ is independently H or optionally substituted alkyl; each of R^{5a} and R^{5b} is independently H, optionally substituted alkyl, optionally substituted alkyl, optionally substituted alkyl, optionally substituted aryl, or optionally substituted heteroaryl; R⁶ is H or optionally substituted alkyl; and m is 1, 2, 3, 4, or 5.

In some embodiments, R¹ is H. In some embodiments, each of R^{2a} and R^{2b} is independently H. In some embodiments, m is 1. In some embodiments, each of R³ and R⁴ is H. In some embodiments, each of R^{5a} and R^{5b} is independently H. In some embodiments, R6 is H. In some embodiments, the compound of Formula (V) is a phosphocreatine. In some embodiments, the compound of Formula (V) is Formula (V-a):

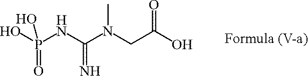

In some embodiments, the compound of Formula (V) is phosphocreatine (e.g., Formula (V-a).

In some embodiments, the adhesive composition comprises an organic compound of Formula (VI):

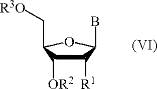

or a salt thereof, wherein B is a nucleobase; R¹ is H, OR⁴, or halo; R² is H, optionally substituted alkyl, optionally substituted alkenyl, optionally substituted aryl, optionally substituted heteroaryl, optionally substituted cycloalkyl, or optionally substituted heterocyclyl; R³ is H, optionally substituted alkyl, or a phosphate moiety (e.g., monophosphate or diphosphate); and R⁴ is H, optionally substituted alkyl, optionally substituted alkenyl, optionally substituted aryl, optionally substituted heteroaryl, optionally substituted cycloalkyl, or optionally substituted heterocyclyl.

In some embodiments, B is a naturally occurring nucleobase or a non-naturally occurring nucleobase. In some embodiments, B comprises adenine, cytosine, guanosine, thymine, or uracil. In some embodiments, each of R¹, R², and R³ is H. In some embodiments, R3 is a phosphate group, e.g., a monophosphate, diphosphate, or triphosphate. In some embodiments, the compound of Formula (VI) is Formula (VI-a) or (VI-b):

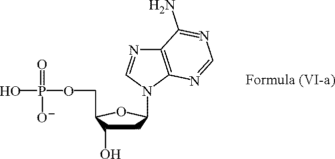 [SMILES: Nc1ncnc2c1ncn2[C@H]1C[C@@H](O)[C@@H](COP(=O)([O-])O)O1]

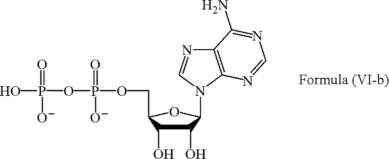

For example, the compound of Formula (VI) is 2′-deoxyadenosine monophosphate or 2′-deoxyadenosine diphosphate.

In some embodiments, the adhesive composition further comprises an additive. The additive may comprise a salt (e.g., calcium carbonate, calcium bicarbonate, sodium carbonate, sodium bicarbonate, sodium chloride, potassium chloride). The additive may comprise one or more of a filler, a formulation base, an abrasive (e.g., bone fragment), a coloring agent (e.g., dye, pigment, or opacifier), a flavoring agent (e.g., sweetener), or a polymer. The additive may comprise a viscosity modifier (e.g., a polyol (e.g., glycerol, mannitol, sorbitol, trehalose, lactose, glucose, fructose, or sucrose)). The additive may comprise a medication that acts locally (e.g., anesthetic, coagulant, clotting factor, chemotactic agent, and agent inducing phenotypic change in local cells or tissues), a medication that acts systemically (e.g., analgesic, anticoagulant, hormone, enzyme co-factor, vitamin, pain reliever, anti-inflammatory agent, chemotactic agent, or agent inducing phenotypic change in local cells or tissues), or an antimicrobial agent (e.g., antibacterial, antiviral, or antifungal agent).

In some embodiments, the adhesive composition further comprises an aqueous medium (e.g., water or saline).

The adhesive composition described herein may be self-setting or light-cured upon activation. In some embodiments, the adhesive composition may be mixed at the time of use with an activator, e.g., an aqueous medium to initiate self-setting. In some embodiments, said aqueous medium may comprise a blood-based product, water, or other aqueous medium biocompatible with bodily fluids. In an embodiment, said aqueous medium comprises water. To form an exemplary adhesive composition, for example, tetracalcium phosphate and phosphoserine may be mixed with water. In some embodiments, the combined weight of the multivalent metal compound and the organic-based compound comprises between about 10% to 90% of the total composition. In an embodiment, the aqueous medium comprises about 35% of the total composition. In some embodiments, the composition may comprise an additional additive, such as a salt, a filler, a viscosity modifier, an antibiotic, or a medication.

In some embodiments, the adhesive composition disclosed herein is bioresorbable, e.g., that disperses and is replaced with native tissue over time. In some embodiments, the adhesive composition is formed by mixing an organic compound (e.g., a compound of Formulas (I), (II), (III), (IV), (V), or (VI)) and multivalent metal salt with an aqueous medium. In some embodiments, said adhesive composition may be applied in its fluid or semi-solid state to the surface of a device or flap by means of an injection delivery device or similar application means. In some embodiments, the adhesive composition may have a tacky state for 5 to 20 minutes upon activation through mixing with the aqueous medium. In some embodiments, the adhesive composition may have a tacky state for approximately 12 minutes upon mixing. In some embodiments, during said tacky state, the adhesive composition may have a tack strength to surfaces (e.g., bone, metal, plastic) of between about 10 kPa and about 250 kPa. In some embodiments, the adhesive composition has a putty state for between 10 and 20 minutes after the tacky state. In some embodiments, the adhesive composition has a putty state for about 15 minutes. In some embodiments the tack stress in the putty state is between about 10 kPa and about 250 kPa. In some embodiments, upon curing, the adhesive composition may have an adhesive strength to surfaces (e.g., bone, metal, plastic) of greater than 1000 kPa.

In one embodiment, the disclosure features a flap comprising an adhesive composition. In some embodiments, the adhesive composition may comprise a biomaterial or combination of biomaterials. In some embodiments, said biomaterial comprises an adhesive composition. In some embodiments, the flap comprises a solidified form of an adhesive composition. The device may be substantially comprised of the adhesive composition, or may comprise additional components (e.g., a fiber). Exemplary flaps may comprise an additional layer of the adhesive composition (e.g., in the working state) as a coating on the surface of the device or impregnated into or onto the surface of the adhesive device or into the kerf space where the flap is placed into is original or corrected position. In some embodiments, an additional layer of the adhesive composition is partially or fully filled into the kerf space to fixate the flap (e.g., cranial flap). In some embodiments, the flap and an additional layer of the adhesive composition is used to block the flow of an aqueous medium. In some embodiments, the flap and an additional layer of the adhesive composition is used to reinforce a structure (e.g., cranium or spine). In some embodiments, the flap and an additional layer of the adhesive composition is used to join separated objects. In other embodiments, the flap and an additional layer of the adhesive composition is used for filling of space to connect and immobilize a structure. In still other embodiments, the flap and an additional layer of the adhesive composition may be used in a method of treating a subject suffering from a disease or condition.

### Methods of Use

The present disclosure features various methods of use for the devices and compositions described herein. In some embodiments, the devices and methods of use described herein may improve cosmetic appearance by: increased level positioning of the flap; prevention of tissue sagging into the osteotomy, i.e., burr holes, kerf lines; or by elimination of hardware protrusion, e.g., resting above the bony contour. In some embodiments, elimination of hardware protrusion reduces palpability and soft tissue irritation over time, and therefore causes reduction in pain.

In some embodiments, the devices and methods of use described herein create a watertight seal of the osteotomy, i.e., burr holes or kerf lines, and thereby aide in the prevention of hydrodynamic complications e.g., leaks of cerebrospinal fluid out of, or bacterial penetration into, the cranial cavity. The creation of a watertight seal reduces the occurrence of secondary infections.

In some embodiments, the devices and methods of use described herein improve the fixation strength of the flap and relative motion of the flap to the host bone as compared the current standard of care, e.g., plates and screws, by providing a structural scaffold in the osteotomy that bonds the flap and host bone. In some embodiments, said structural scaffold comprises a resorbable biomaterial and replaced by native material over time.

In some embodiments, the devices described herein does not comprise hardware, e.g., plates and screws and thus reduce the occurrence of secondary infections by the elimination of such hardware. In some embodiments, the devices and methods of use described herein eliminates or significantly reduces CT or MRI artifacts by the elimination of metal hardware.

In some embodiments, the devices and methods of use described herein creates a seal around the osteotomy, i.e., burr holes and kerf lines, which can prevent fibrous tissue ingrowth. In some embodiments, this seal may also facilitate bone fusion, which provides the bone flap with a vascular supply and therefore prevents resorption in size of the flap, e.g., change in thickness and width. In some embodiments, the devices, compositions and methods of use described herein improves flap placement, fixation strength and prevent migration of the flap.

In some embodiments, a plurality of flap spacer devices (FSD)s may be distributed spaced out over the run of the osteotomy, e.g., a craniotomy, with the projections (P) extending into the kerf space to provide a roughly uniform separation between the edges of the flap to be affixed and the cut edge of the skull (FIG. 2). In some embodiments, this means of placing and fixating the flap in situ allows the optional placement of a flap intraoperative retainer device (FR) to further immobilize the flap, as needed (FIG. 3). In some embodiments, (FR) is rigidly fixated to the skull and rigidly retains the flap in the desired position so that an adhesive composition can be delivered to the kerf space and allowed to cure undisturbed by flap movement (FIG. 4). In some embodiments, once the extent of curing is sufficient, (FR) is detached from the flap and the skull, leaving the flap rigidly attached in place. In some embodiments, (FSD)s might then be removed, and the spaces vacated by their removal from the kerf space may be filled with an adhesive composition. In some embodiments, the projections (P) of the (FSD)s may remain, with or without the remainder of the (FSD) remaining in situ following the completion of the procedure.

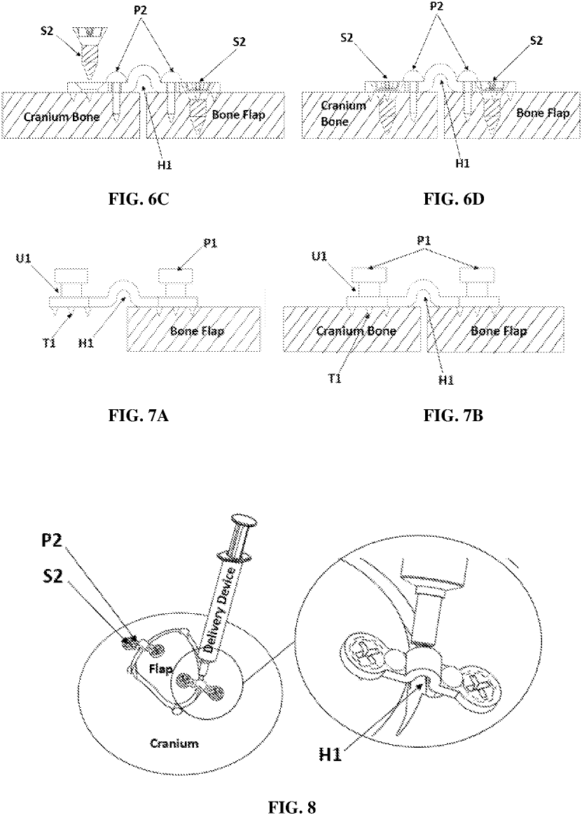

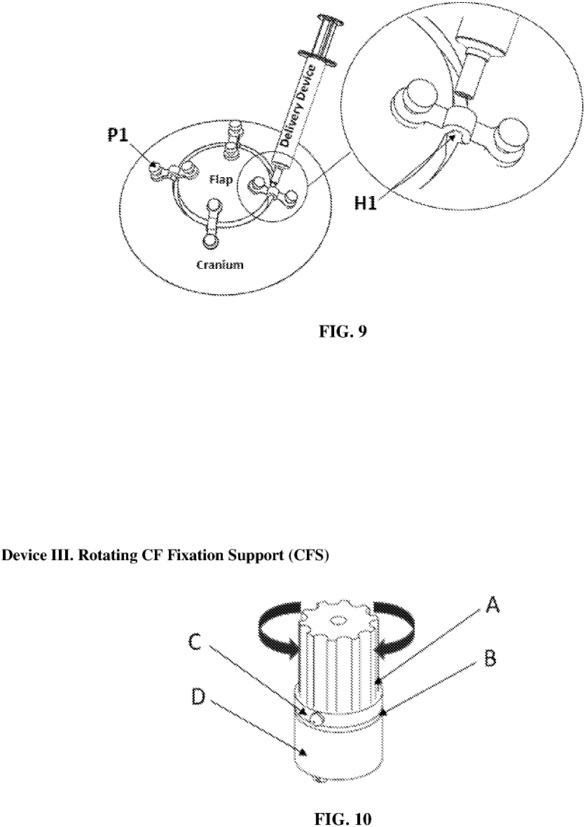

The present disclosure also features methods for use wherein a plurality of flap retainer-with-relief devices (FRR)s may be displaced about the run of the osteotomy e.g., craniotomy. In some embodiments, kerf spanning relief (H1) may be aligned with the center of the kerf width to provide a roughly uniform separation between the edges of the flap to be affixed and the cut edge of the skull (FIG. 6). In some embodiments, this means of placing and fixating the flap in situ then allows for the optional placement of one or more (FRR)(s) to further immobilize the flap. In some embodiments, said (FRR)s may be fixated using a mallet to compress protrusions (T1) into the native bone and flap. In some embodiments, (FRR)s are fixated using a mallet to compress pins or nails (P1) into the native bone and flap. In some embodiments, said (FRR)s are fixated using pre-drilled holes to allow for screws (S2) to be threaded into the native bone and flap. In some embodiments, said (FRR) are rigidly fixated to the native bone once aligned with the kerf line and rigidly retain the flap in the desired position so that an adhesive composition is delivered to the kerf space and allowed to cure undisturbed by flap movement (FIGS. 8-9). In some embodiments, once the extent of curing is sufficient to support the flap, (FRR)s are detached from the flap and the native bone, leaving the flap rigidly attached in place and (FRR)s are then removed. In some embodiments the attachment spaces vacated by the (FRR) removal from the kerf space are filled with the adhesive composition. In some embodiments, the kerf spanning relief (H1) allows for separation creating two equal halves thereby allowing each section to be removed separately.

The present disclosure features a further method of use wherein a plurality of (RFS)s may be spaced about the run of the osteotomy, e.g., craniotomy (FIG. 10). In some embodiments, foot (F) of the device may be aligned with the center of the kerf width to provide a roughly uniform separation between the edges of the flap to be affixed, e.g. a cranial flap, and the cut edge of the native bone (FIG. 11). In some embodiments, this allows for placing and fixating the flap in situ and optional placement of multiple (RFS)s to further immobilize the flap, if required. In some embodiments, once the flap is placed into position (RFS) is inserted into relevant positioning by placing toggle assembly (J) along the kerf line then compressing the handle to allow the deformation of compression washer (D) thereby extending the reach of foot (F) depending on thickness of the native bone. In some embodiments, once foot (F) has been extended past the native bone thickness, knob (K) isrotated to allow foot (F) to make contact with the native bone and flap (FIG. 12). In some embodiments, once the thumb handle with alignment markers (H) has shown to be in contact with the native bone and flap, handle (H) may be released thereby providing a compressive load onto the upper and lower surface of the flap and native bone and fixing the flap in place in proper alignment (FIG. 13). In some embodiments, once alignment and fixation have been achieved, an adhesive composition is delivered. In some embodiments, once the adhesive composition has set, each (RFS) is removed by compressing handle (H) and rotating until foot (F) is aligned with the kerf space; the device is removed by gently pulling foot (F) free of the kerf space. In some embodiments, upon removal of the (RFS), the adhesive composition isdelivered to the remainder of the kerf space to form a complete seal.

The present disclosure also features a method of use wherein a plurality of compressive support screws (CSS) may be distributed spaced out over the run of the osteotomy, e.g., a craniotomy (FIG. 16). In some embodiments, transition holes are placed that are larger than the width of the kerf space that have been aligned with the center of the kerf width, to provide a roughly uniform separation between the edges of the flap to be affixed and the cut edge of the native bone (FIG. 16). In some embodiments, once the flap is placed in position, each (CSS) may be inserted into relevant position in the kerf space by positioning expandable protrusion (EP) in the center of the width of the kerf or burr hole. In some embodiments, once expandable protrusion (EP) has been placed, threaded screw (S1) is rotated in order to provide pressure on (EP), said pressure expanding (EP) to support and fixate the flap in its position. In some embodiments, support surface (SS) holds the flap and native bone in their positions once expandable protrusion (EP) has been engaged. In some embodiments, once alignment and fixation have been achieved, an adhesive composition is delivered. In some embodiments, once said adhesive composition has set, each (CSS) is removed by rotating threaded screw (S1) to decompress expandable protrusion (EP), thus relieving the pressure on support surface (SS), after which the device is removed. In some embodiments, upon removal of each (CSS) the adhesive composition is delivered to the gaps left by the removed (CSS)s, to completely seal the kerf.

A further embodiment of the present disclosure features a method of use featuring bone flap articulating arm (BFA). In some embodiments, clamp (A) may be attached to a work surface by compressing the arm clamp until the span (CD) allows for each clamp arm to fully capture the supporting surface (FIG. 17). In some embodiments, once clamp (A) is in location the clamp arms may be released allowing the clamp arms to close and capture the surfaces using an integral spring. The articulating arm (C) may be attached using (B1) which allows arm to be fixed in position but also manipulatable. The flap may be attached to the primary alignment protrusion (B2) using screws, protrusions, hook and loop, vacuum cup, temporary gel adhesive, or other similar such method. In some embodiments, once the flap has been attached, it may then be mated to the distal end of articulating arm (C) which provides the flap freedom to be manipulated. In some embodiments, while the flap is attached to it, it may be manually manipulated into position, with (BFA) aligned in the center of the kerf width to provide a roughly uniform separation between the edges of the flap to be affixed and the cut edge of the native bone. In some embodiments, once alignment and fixation has been achieved an adhesive composition is delivered (FIG. 18). In some embodiments, once the adhesive composition has set the arms are detached from the flap.

The present disclosure features a further embodiment, wherein a plurality of (KFFD)s may be distributed spaced out over the run of the osteotomy, e.g., a craniotomy. In some embodiments, there may be transition holes which are larger than the width of the kerf and which have been aligned with the center of the kerf width, thus providing a roughly uniform separation between the edges of the flap to be affixed and the cut edge of the native bone. In some embodiments, once the flap is placed in position said (KFFD)s may be inserted into proper position by placing support foot distal (SFD) into transition holes created prior to the kerf being cut (FIG. 21). In some embodiments, after each device has been placed, support foot distal (SFD) is rotated 30-90 degrees opposed to the kerf line; support foot distal (SFD) is then placed over the flexible loop (FL) and flexible shaft (FS) until it is compressed onto the transitions joint (TJ) of the support foot distal (SFD) (FIG. 20). In some embodiments, a locking mechanism (LM) is utilized to keep support foot distal (SFD) in place by holding the flap in position (FIG. 22). In some embodiments, once alignment and fixation have been achieved an adhesive composition is delivered. In some embodiments, once the adhesive composition has set, each (KFFD) is removed, and an adhesive composition is delivered to the spaces where each (KFFD) had been, to fully fill the kerf space.

The present disclosure features a further embodiment, wherein a plurality of flap step fixation devices (FSF)s may be distributed, spaced out over the run of the osteotomy, e.g., craniotomy. In some embodiments, transition holes have been created that are larger than the width of the kerf space (FIG. 25). In some embodiments, said transition holes are aligned with the center of the kerf space width in order to provide a roughly uniform separation between the edges of the flap to be affixed and the cut edge of the native bone. In some embodiments, each (FSF) is placed in transition holes and aligned to be parallel to the kerf space using alignment feature (KA) (FIG. 24). In some embodiments, a screw (CS) is placed in the center of the device, and wherein the screw (CS) is rotated while holding the device in place. In some embodiments, said screw stresses the outer wall of (FSF)until it expands, thus providing support along the transition hole’s inner diameter. In some embodiments, once alignment and temporary fixation has been achieved an adhesive composition is delivered. In some embodiments, once the adhesive composition has set, each (FSF) is removed, and an adhesive composition is delivered to the spaces where each (FSF) had been, to fully fill the kerf space.

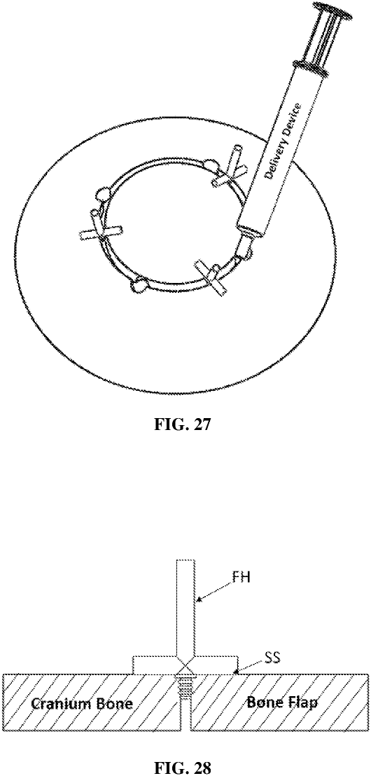

In a further embodiment a plurality of bone flap alignment wedge devices (BFW)s may be distributed into the kerf space between the edges of the flap to be affixed and the cut edge of the native bone. In some embodiments, (BFW)s are placed into the kerf space next to, not necessarily into, any surgically created burr holes (FIG. 27). Each device may be placed into position by gently depressing the grabbing surface (GS) into the kerf until the support surface (SS) is aligned to keep the flap surface and native bone parallel to each other (FIG. 28). Once the flap is aligned and fixated an adhesive composition is delivered to the kerf space (FIG. 27). Once said adhesive composition has set, each (BFW) is removed by rotating said device and gently pulling it out of the kerf space. Upon removal of said devices, the adhesive composition is delivered to the areas where the devices had been, in order to fully fill the kerf space.

The above methods of use as described herein may be used solely or in combination, or possibly in addition to other methods of use.

### ENUMERATED EMBODIMENTS

1. A device for positioning or fixating a flap in or on a bone, wherein (i) the device holds the flap in proximity to the bone to allow for adhering the flap to the bone with an adhesive composition and (ii) the device does not comprise a bone screw, bone plate, connecting suture, or connecting wire.

2. The device of embodiment 1, wherein the device is part of an assembly of devices for positioning or fixating a flap in a bone.

3. The device of any one of embodiments 1-2, wherein the flap is a cranial flap in a subject.

4. The device of any one of embodiments 1-3, wherein the bone is the cranium of a subject.

5. The device of any one of embodiments 1-4, wherein the device or assembly of devices is only used for a finite duration of time, e.g., is not permanently adhered to or implanted into a subj ect.

6. The device of any one of embodiments 1-5, wherein the device or assembly of devices protrudes from the surface of the bone by about 1 mm, 2 mm, 3 mm, 5 mm, 7.5 mm, 10 mm, or more.

7. A device for positioning or fixating a flap in a bone, wherein the device comprises a means for adhering the flap to the bone with an adhesive composition.

8. A device for positioning or fixating a flap in a bone, wherein the device provides a means for fixation without use of a bone screw, bone plate, connecting suture, or connecting wire.

9. A device for positioning or fixating a flap in a bone, wherein: (i) the device comprises a means for adhering the flap to the bone with an adhesive composition and/or; (ii) the device comprises an adhesive composition.

10. A device for positioning or fixating a flap in a bone, wherein: (i) the device comprises a means for adhering the flap to the bone with an adhesive composition and/or; (ii) the device provides a means for administration of an adhesive composition to the space (e.g., the kerf space) between the flap and the bone.

11. The device of embodiment 9, comprising (i).

12. The device of embodiment 9, comprising (ii).

13. The device of embodiment 10, comprising (i).

14. The device of embodiment 10, comprising (ii).

15. The device of any one of embodiments 1-14, wherein the adhesive composition comprises a biomaterial.

16. The device of any one of embodiments 1-15, wherein the flap in the bone is surgically created or is created from trauma.

17. The device of any one of embodiments 1-16, wherein the flap comprises native bone, xenograft bone, allograft bone, or a synthetic biomaterial.

18. The device of any one of embodiments 1-17, wherein the device further comprises a biocompatible material comprising native bone, xenograft, allograft; or a synthetic biocompatible material.

19. The device of any one of embodiments 1-18, wherein the device comprises a synthetic biocompatible material.

20. The device of any one of embodiments 18-19, wherein the synthetic biocompatible material comprises one or more of: steel or steel alloy, titanium or titanium-based alloy; a plastic material; an inorganic material such as hydroxyapatite, or alumina ceramics; organic materials such as transplanted bone, or organic composite; and combinations thereof.

21. The device of any one of embodiments 1-20, wherein the device is of sufficient strength to withstand placement, adjustment and removal of said device, other devices, or flap without compromising the fixated position of the flap.

22. The device of any one of embodiments 1-21, wherein said device comprises a resorbable biomaterial.

23. The device of embodiment 22, wherein said resorbable biomaterial is dispersed and replaced by host tissue over time.

24. The device of any one of embodiments 1-23, further comprising a means for allowing a user to position and fixate a flap, e.g., in its original or corrected position.

25. The device of embodiment 24, wherein said means is temporary or definitive.

26. The device of any one of embodiments 24-25, wherein said means comprises a mechanical fixation feature.

27. The device of any one of embodiments 1-26, wherein said device further comprises a mechanical feature usable within or across the space between the flap and the bone (e.g., the kerf space) dimension and bone thickness.

28. The device of any one of embodiments 1-27, wherein the device comprises a protrusion (e.g., wherein said protrusion may be pressed into the surface of native bone and/or flap using a mallet or other compressive methods to allow for temporary fixation of the flap in its original or corrected position).

29. The device of embodiment 28, further comprising a clamp.

30. The device of embodiment 29, wherein said clamp further comprises a supportive structure.

31. The device of embodiment 30, wherein the supportive structure comprises a compressive material.

32. The device of embodiment 31, wherein the compressive material comprises any soft durometer material or spring-like material, e.g., to allow for fixation of the flap in its original or corrected position.

33. The device of any one of embodiments 1-32, wherein the device uses a compressive force (e.g., opposing compressive forces) to align and fixate the flap in its original or corrected position.

34. The device of embodiment 33, comprising vacuum suction forces for fixating the flap into its original or corrected position.

35. The device of any one of embodiments 1-35, wherein the device further comprises a cam design (e.g., rotating cam design).

36. The device of embodiment 35, wherein said cam design engages compressive forces when rotated to temporarily fixate the flap in its original or corrected position.

37. The device of any one of embodiments 1-36, wherein the device further comprises a durometer material, e.g., to apply compressive forces against the flap and bone to temporarily fixate the flap in its original or corrected position.

38. The device of any one of embodiments 1-37, wherein the device further comprises a friction lock, e.g., to adjust alignment and lock the flap in its original or corrected position.

39. The device of any one of embodiments 1-38, wherein the device further comprises a threaded screw, e.g., to adjust and lock the flap in its original or corrected position.

40. The device of any one of embodiments 1-39, wherein said device creates a parallel surface between the flap and the native bone, e.g., to ensure the flap is in its native height and position.

41. The device of any one of embodiments 1-40, wherein the device further comprises a structure to be bound to the bone via a screw, rod, plate, suction, or adhesive bonding.

42. The device of any one of embodiments 1-41, wherein the device further comprises an adjustable Z-axis.

43. The device of any one of embodiments 1-42, wherein the device further comprises a support structure.

44. The device of embodiment 43, wherein the support structure attaches to a section of the device from where a plurality of structures may be attached.

45. The device of any one of embodiments 1-44, further comprising a screw relief hole.

46. The device of embodiment 45, wherein said screw relief hole comprises one or more sizes.

47. The device of any one of embodiments 1-46, wherein said adhesive composition comprises a multivalent metal salt, an organic compound, and an aqueous medium.

48. The device of embodiment 47, wherein said multivalent metal salt is selected from the group consisting of calcium phosphate, tricalcium phosphate (e.g., alpha tricalcium phosphate or beta tricalcium phosphate), tetracalcium phosphate, and mixtures thereof.

49. The device of any one of embodiments 47-48, wherein said multivalent metal salt comprises tetracalcium phosphate.

50. The device of any one of embodiments 47-49, wherein said organic compound is selected from the group consisting of a compound of Formulas (I), (II), (III), (IV), (V), and (VI), e.g., phosphoserine, carboxy ethyl phosphonate, phosphonoacetic acid, and mixtures thereof.

51. The device of any one of embodiments 47-50, wherein said organic compound comprises a compound of Formula (IV), e.g., phosphoserine.

52. The device of any one of embodiments 47-51, wherein said aqueous medium is selected from the group consisting of water or a blood-based product.

53. The device of any one of embodiments 41-52, wherein said aqueous medium is water.

54. The device of any one of embodiments 1-53, wherein said adhesive composition is activated upon mixing the components with said aqueous medium (e.g., water).

55. The device of any one of embodiments 1-54, wherein upon activation of the adhesive device with the aqueous medium, said adhesive composition has an initial tacky state for up to about 2 minutes after mixing.

56. The device of any one of embodiments 1-55, wherein said adhesive composition during said tacky state has a separation strength of about 50 kPa to about 150 kPa after mixing.

57. The device of any one of embodiments 1-56, wherein said adhesive composition has a putty state for about 8 minutes after mixing with the aqueous medium.

58. The device of any one of embodiments 1-57, wherein said adhesive composition has a working time of about 7 to about 12 minutes after mixing with the aqueous medium.

59. The device of any one of embodiments 1-58, wherein said adhesive composition has an adhesive strength upon curing of 100 kPa or greater (e.g., 250 kPa, 500 kPa, 750 kPa, 1,000 kPa, 1,250 kPa, 1,500 kPa, 2,000 kPa, 2,500 kPa, or more).

60. The device of any one of embodiments 47-59, wherein said multivalent metal salt and said organic compound are each independently present in the adhesive composition in an amount from about 10% to about 90% (e.g., 10% to 75%, 25 to 50%) w/w, w/v, or v/v of the total weight of all components in the adhesive composition.

61. The device of any one of embodiments 47-60, wherein said aqueous medium is present in an amount up to about 10% or more, e.g., 15%, 20%, 25%, 30%, or 35%, w/w, w/v, or v/v of the total weight of all components in the adhesive composition.

62. The device of any one of claims 1-61, wherein said adhesive composition further comprises an additive, e.g., a salt, filler, viscosity modifier, an antibiotic, or other medication.

63. The device of any one of embodiments 1-62, wherein said adhesive composition fixates the flap in its original or corrected position.

64. The device of any one of embodiments 1-63, wherein said adhesive composition partially or fully fills the space between the flap and the bone, e.g., the kerf.

65. The device of any one of embodiments 1-64, wherein said adhesive composition seals the kerf, e.g., against leakage or infection.

66. The device of any one of embodiments 1-65, wherein said device is engaged in methods to definitively fixate the flap, e.g., in its original or corrected position.

67. An assembly of devices for positioning or fixating a flap in or on a bone, wherein the assembly comprises at least 2, 3, 4, 5, 6, 7, 8, or devices, e.g., devices of any one of embodiments 1-66.

68. A method of fixating a flap (e.g., a bone flap), e.g., in its original or corrected position, by placing a device of any one of embodiments 1-66 or an assembly of devices of embodiment 67.

69. The method of embodiment 68, wherein said device comprises a means for delivering an adhesive composition.

70. The method of any one of embodiments 68-69, wherein utilization of the device eliminates hardware protrusion.

71. The method of embodiment 70, wherein the device protrudes from the surface of the bone by more than 0.5 mm, 1 mm, 2 mm, 3 mm, 4 mm, 5 mm, 7.5 mm, 10 mm, or more.

72. The method of any one of embodiments 70-71, wherein eliminating hardware protrusion reduces palpability and soft tissue irritation, e.g., compared with palpability and soft tissue irritation from use of a bone screw, bone plate, connecting wire, or connecting suture.

73. The method of any one of embodiments 68-72, wherein said device further comprises a structural scaffold, e.g., to be placed in the osteotomy.

74. The method of embodiment 73, wherein said structural scaffold bonds to the flap and bone (e.g., native bone).

75. The method of any one of embodiments 68-74, wherein a plurality of devices is distributed into the kerf space (e.g., wherein said devices are placed partly within the kerf to provide roughly uniform separation between the edge of the flap and the cut edge of the native bone).

76. The method of any one of embodiments 68-75, wherein said device is fixated into the kerf via protrusions, compression, screws, locking mechanisms and other such mechanisms.

77. The method of any one of embodiments 68-76, further comprising use of an articulating arm, e.g., to manipulate the flap into its original or corrected position for definitive fixation.

78. The method of any one of embodiments 68-77, wherein a bone substitute is delivered to the kerf space.

79. The method of embodiments 78, wherein said bone substitute is a biomaterial or combination of biomaterials.

80. The method of embodiment 79, wherein said biomaterial comprises an adhesive composition.

81. The method of any one of embodiments 68-80, wherein said device comprises a malleable material, e.g., to allow manual adjustment of flap alignment.

82. The method of any one of embodiments 68-81, wherein said device remains in the kerf space during delivery of the adhesive composition.

83. The method of any one of embodiments 68-82, wherein said device remains in the kerf space during curing of the adhesive composition.

84. The method of any one of embodiments 68-83, wherein said device is removable from the kerf space.

85. The method of any one of embodiments 68-84, wherein said device remains in the kerf during placement of an additional device or additional treatment.

86. The method of any one of embodiments 68-85, wherein said device remains in the kerf space such that an adhesive composition may be fully delivered into the kerf space creating a seal, e.g., against leakage.

87. The method of any one of claims 68-86, wherein said device comprises a resorbable biomaterial.

88. The method of embodiment 87, wherein said resorbable material disperses and is replaced with host tissue over time.

89. The device of any one of embodiments 1-66, or the assembly of devices of embodiment 67, wherein the device comprises any one of a device selected from RFS, FSD, FR, FRR, CSS, BFA, KFFD, FSF, BFW, CFS, and CFFS, e.g., as described herein.

90. The device of any one of embodiments 1-66, or the assembly of devices of embodiment 67, wherein the device comprises RFS, e.g., as described herein.

91. The device of any one of embodiments 1-66, or the assembly of devices of embodiment 67, wherein the device comprises FSD, e.g., as described herein.

92. The device of any one of embodiments 1-66, or the assembly of devices of embodiment 67, wherein the device comprises FR, e.g., as described herein.

93. The device of any one of embodiments 1-66, or the assembly of devices of embodiment 67, wherein the device comprises FRR, e.g., as described herein.

94. The device of any one of embodiments 1-66, or the assembly of devices of embodiment 67, wherein the device comprises CSS, e.g., as described herein.

95. The device of any one of embodiments 1-66, or the assembly of devices of embodiment 67, wherein the device comprises BFA, e.g., as described herein.

96. The device of any one of embodiments 1-66, or the assembly of devices of embodiment 67, wherein the device comprises KFFD, e.g., as described herein.

97. The device of any one of embodiments 1-66, or the assembly of devices of embodiment 67, wherein the device comprises FSF, e.g., as described herein.

98. The device of any one of embodiments 1-66, or the assembly of devices of embodiment 67, wherein the device comprises BFW, e.g., as described herein.

99. The device of any one of embodiments 1-66, or the assembly of devices of embodiment 67, wherein the device comprises CFS, e.g., as described herein.

100. The device of any one of embodiments 1-66, or the assembly of devices of embodiment 67, wherein the device comprises CFFS, e.g., as described herein.

101. A method of treating a subject having an injury, disease or disorder, e.g., an injury, disease or disorder described herein, comprising using a device or an assembly of devices, e.g., described herein, for positioning or fixating a flap in or on a bone in a subject, thereby treating the injury, disease or disorder.

### EXAMPLES

### Example 1: Exemplary Use of Rotating Fixation Device (RFS)

An exemplary rotating flap fixation device (RFS), as described herein, may be used in a surgery to temporarily locate a flap in position while allowing spot or continuous injection of an adhesive composition with or without need for removal. A surgeon will cut the surgical area, e.g., cranium, to perform the necessary procedure, leaving a detached flap and kerf space. The device or series of devices will be presented to the surgical field and placed along flap surface by: compressing spring (D) allowing foot (F) to span the flap thickness (FE) and then rotating and releasing pressure on rotating handle (A) to attach the device to the flap by the force provided by the semi-loaded spring (D) or soft durometer compression washer (D) whereby squeezing the surface of (SB) and the foot (F). Upon completion of the procedure, the flap with each device secured in place will be gently introduced, causing rotating surface (SB) of said devices to contact the native bone whereby aligning the surfaces of each, with any small adjustments being made at time of placement. An adhesive composition will be delivered continuously or in a spot fashion around the kerf space to definitively fixate the flap. Said adhesive composition comprises a multivalent metal salt, an organic compound, and an aqueous medium. The multivalent metal salt and organic compound will be mixed with the aqueous medium, which serves as an activator. Upon mixing, the composition develops a tacky and then a putty state, with a working time of about 5 to 12 minutes during which the surgeon may deliver the adhesive composition into the kerf space. Once the adhesive composition has been delivered and sufficiently cured, the (RFS)s may be removed followed by further application of the adhesive composition to form a complete seal. The placement and securing of said (RFS)s allows for continuous or spot delivery of an adhesive composition to the kerf space to fixate the flap. Optionally, flap spacer devices (FSD)s may be spaced throughout the kerf space in addition to utilizing said (RFS)s, wherein (FSD)s provide greater fixation support.

### Example 2: Exemplary Use of Flap Spacer Device

An exemplary flap spacer device (FSD), as described herein, may be used in a cranial surgery in order to position and fixate a cranial flap (FIGS. 1-2). Once a surgeon has completed an osteotomy and the cranial flap is primed for placement, a plurality of (FSD)s will be prepared for temporary fixation within the surgical site using aseptic technique. Each (FSD) will be fixated in the center of the kerf space between the flap to be fixated and the native bone such that flange (R) maintains contact with the flap and native bone and limits movement of projection (P). A plurality of (FSD)s will be placed to maintain a consistent kerf line width across the osteotomy. The surgeon will place the devices by: gripping flange (R) and placing projection (P) into the kerf space. The placement of said devices will provide roughly uniform spacing for the placement of another device, e.g., another device described herein, or the delivery of anadhesive composition. Once the adhesive composition has been delivered and cured, (FSD)s will be removed and adhesive composition delivered to the spaces where they had been located.

### Example 3: Exemplary Use of Flap Retainer With Relief (FRR)

An exemplary flap retainer with relief (FRR), as described herein, may be used in a surgery to temporarily fixate a flap in position while allowing continuous injection of an adhesive composition without need for removal. A surgeon will cut the surgical area, e.g., cranium, to perform the necessary procedure, leaving a detached flap and kerf space. Upon completion of the procedure, multiple flap retainers with relief (FRR) will be placed into the surgical site with: kerf spanning relief (H1) spanning the kerf space; and protrusions (T1) that allow the device to be secured into the native bone and the flap to be fixated on either side of the kerf space (FIGS. 6A-D and 7A-B). Optionally, said protrusions (T1) may be replaced with screws to secure the device. Once each device is secured and the flap is fixated in position, an adhesive composition will be delivered continuously around the kerf space to definitively fixate the flap. Said adhesive composition comprises a multivalent metal salt, an organic compound, and an aqueous medium. The multivalent metal salt and organic compound will be mixed with the aqueous medium, which serves as an activator. Upon mixing, the composition develops a tacky and then a putty state, with a working time of about 7 to 12 minutes during which the surgeon may deliver the adhesive composition into the kerf space. Once the adhesive composition has been delivered and cured, the (FRR)s may be removed. The placement and securing of said (FRR)s allows for continuous delivery of an adhesive composition to the kerf space to form a complete seal, wherein removal of said devices will not disrupt said seal. Optionally, flap spacer devices (FSD)s may be spaced throughout the kerf space in addition to utilizing said (FRR)s, wherein (FSD)s provide greater fixation support.

### Example 4: Exemplary Adhesive Compositions for Use With a Device

Exemplary adhesive compositions for use with a flap fixation device to secure a flap to native bone have been investigated herein. Listed in Table 1A are exemplary organic compounds that may be incorporated into an adhesive composition as described in Table 1B for use as an adhesive composition to fill in the kerf space and fixate a flap. These adhesive compositions may be used with any of the devices as described in this application (e.g., Example 1 or Example 2, or Example 12 or 13 below). While water was used as the aqueous medium in these exemplary compositions, the aqueous medium may instead be saline, blood, saliva, serum, or a blood-based solution or suspensions. The solid components (i.e., calcium compounds, e.g., tetracalcium phosphate) listed in the table were supplied as particles; however, they may be supplied in granule, or fiber form. In some embodiments, the resulting properties such as working time, setting time, tack strength, and adhesive strength would be affected by these changes. The specific mean particle, granule, or fiber size for each solid component can be selected to satisfy the use requirements. The quantities of each of the components listed may be altered or adjusted in relation to the other components in the composition.

TABLE 1A

Exemplary Organic Compounds

| | | |
|---|---|---|
| Formula | Compound Name | Structure |
| I-a | N,N-Bis(phosphonomethyl)glycine (Glyphosine) | 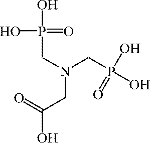 [SMILES: O=C(O)CN(CP(=O)(O)O)CP(=O)(O)O] |
| I-b | (Nitrilotris(methylene))triphosphonic acid (ATMP) | 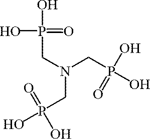 [SMILES: O=P(O)(O)CN(CP(=O)(O)O)CP(=O)(O)O] |
| II-a | Ethylenediamine tetraacetic acid (EDTA) | 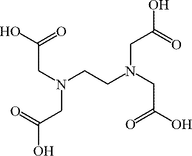 [SMILES: O=C(O)CN(CCN(CC(=O)O)CC(=O)O)CC(=O)O] |
| II-b | Egtazic acid (EGTA) | 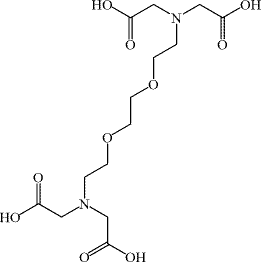 [SMILES: O=C(O)CN(CCOCCOCCN(CC(=O)O)CC(=O)O)CC(=O)O] |
| II-c | Propylenediaminetetraacetic acid (PDTA) | 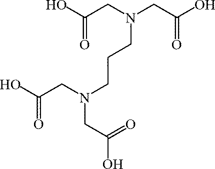 [SMILES: O=C(O)CN(CCCN(CC(=O)O)CC(=O)O)CC(=O)O] |
| III-a | Malonic Acid | 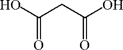 |
| III-b | Citric Acid | 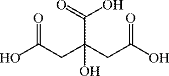 [SMILES: O=C(O)CC(O)(CC(=O)O)C(=O)O] |
| IV-a | O-Phospho-L-Serine (OPLS) | 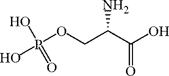 [SMILES: N[C@@H](COP(=O)(O)O)C(=O)O] |
| V-a | Phosphocreatine | 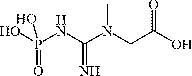 |
| VI-a | 2′-Deoxyadenosine monophosphate | 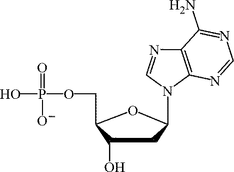 [SMILES: Nc1ncnc2c1ncn2[C@H]1C[C@@H](O)[C@@H](COP(=O)([O-])O)O1] |
| VI-b | 2′-Deoxyadenosine diphosphate | 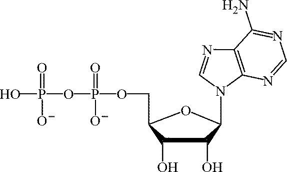 |

TABLE 1B

Exemplary Adhesive Compositions

| Composition | Compound Name | Compound Formula | Compound (mg) | Tetracalcium Phosphate (mg) | Water (µl) |
|---|---|---|---|---|---|
| 1A | Glyphosine | I-a | 711 | 800 | 540 |
| 1B | ATMP | I-b | 202 | 800 | 270 |
| 1C | EDTA¹ | II-a | 395 | 800 | 270 |
| 1D | EDTA² | II-a | 790 | 800 | 370 |
| 1E | EDTA³ | II-a | 550 | 800 | 320 |
| 1F | PDTA | II-c | 414 | 800 | 270 |
| 1G | Malonic Acid | III-a | 281 | 800 | 135 |
| 1H | Citric Acid | III-c | 519 | 800 | 260 |
| 1I | OPLS | IV-a | 500 | 800 | 270 |

### Example 4. Adhesive Shear Strength to Bone Substrate Surfaces

Exemplary adhesive compositions for use with a flap fixation device to secure a flap to native bone have been investigated herein. The shear strength was measured to rupture the bond formed between two bone block surfaces that were adhered together using many of the exemplary adhesive compositions listed in Table 1B. Each composition was prepared by mixing for 20 seconds after addition of the water to ensure a smooth consistency in a 25 mL capacity silicone mixing bowl using a stainless-steel spatula. After mixing, the composition was loaded into a 3 cc capacity slip tip syringe and immediately injected onto one end of each of the 8.5 mm × 8.5 mm surfaces of two rectangular bovine bone blocks that were each 10-15 mm long. Immediately thereafter, the bone block surfaces covered with adhesive composition were apposed and excess material that squeezed out which surrounded the perimeter of the external surfaces of the joint was removed with a spatula. The adjoined blocks were placed into a fixture that applied a slight compressive force of 3 to 5 N for 4 minutes from the start of mixing corresponding to the working period of the compositions from the start of mixing. Thereafter, the blocks were removed from the fixture and submerged into a phosphate buffered saline (PBS) solution bath at 37° C. to allow the compositions to cure for 24 hours from the start of mixing. After curing, the blocks were removed from the PBS bath for shear testing. The proximal block of the adhered block set was secured in a stable fashion to prevent movement within a sample holding fixture up to within 1.0 mm of the adhered joint mounted to an Instron® 5969 axial load frame. The distal block of the adhered block set was cantilevered from the sample holding fixture. The Instron crosshead with an attached anvil fixture was lowered until the distal surface of the anvil was within 0.5 mm of the top surface of the distal bone block and within 1.0 mm of adhered joint. The test was run with the crosshead speed at 2 mm/minute. Table 2 shows the results for the average shear stress (MPa) and standard deviation (MPa) after 24 hours of cure in order to rupture the bond formed at the joint between the adhered bone blocks.

TABLE 2

Adhesive Shear Strength to Rupture Bond of Bone Blocks Adhered Together after Exemplary Adhesive Compositions Cured for 24 Hours in PBS Solution

| Composition | Compound name | Average Shear Stress (MPa), n=3 | Standard Deviation (MPa) |
|---|---|---|---|
| 1A | Glyphosine | 0.37 | 0.09 |
| 1B | ATMP | 0.35 | 0.18 |
| 1C | EDTA¹ | 1.76 | 0.58 |
| 1D | EDTA² | 1.30 | 0.73 |
| 1E | EDTA³ | 2.23 | 0.76 |
| 1F | Propylenediaminetertaacetic Acid | 0.18 | 0.02 |
| 1G | Malonic Acid | 1.68 | 0.54 |
| 1H | Citric Acid | 3.16 | n/a |
| 1I | OPLS | 2.36 | 0.23 |

*n=2 samples tested

### Example 5: Investigation of Flap Fixation Devices

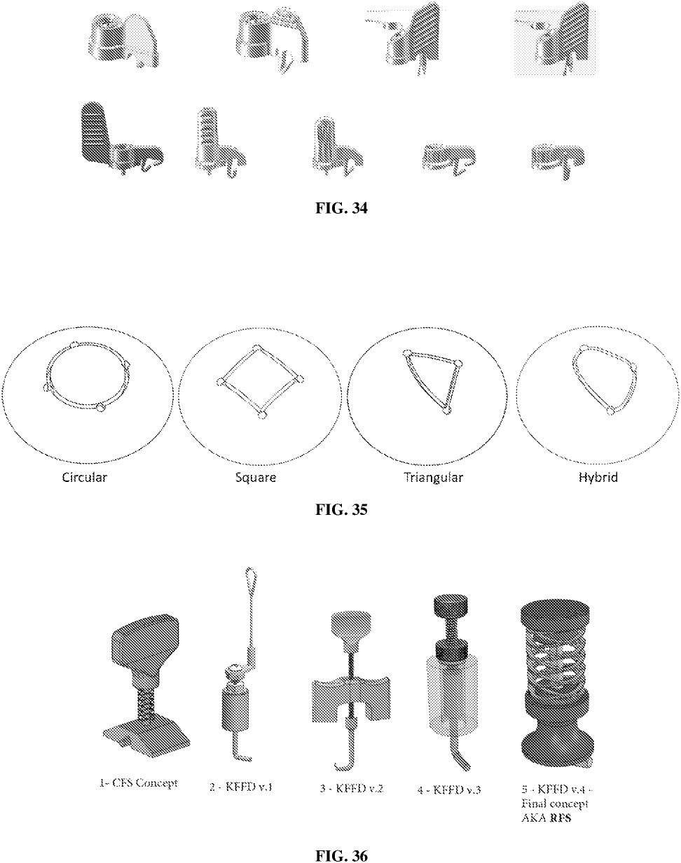

In this example, several devices were investigated to determine which afforded the best overall properties for flap fixation. Various fixation devices were tested and further developed to advance certain features. The devices were evaluated using a concept scoring tool which included the following categories on a scale of 1-5, wherein a score of “1” equates to exceptionally poorly and a score of “5” being equates to exceptionally well: (i) flap holding strength; (ii) z-axis control and alignment; (iii) x- and y-axis control and alignment; (iv) installation and removal usability and speed; (v) adhesive application usability and speed; (vi) cost; and (vii) universal application. There were three essential fixation device designs that were tested and evaluated and further developed according to their higher raw scoring in more important attributes, as can be seen in Table 3: a bone flap wedge (BFW), cranial flap fixation retainer with relief (CFFR), and a rotating CF fixation support (CFS). A kerf flap fixation device (KFFD) was also selected for further development according to surgeon preference. The surgeons were also presented with a cranial flap fixation support (CFFS) which was evaluated and several iterations made according to surgeon feedback. FIGS. 33-34 show exemplary (CFFS) devices and several iterations thereof. FIG. 26 shows an exemplary (BFW), FIGS. 5-7 show an exemplary (CFFR), and FIGS. 10-13 show an exemplary (CFS). FIG. 17 shows an exemplary bone flap articulating arm (BFA). FIGS. 29-32 show exemplary (RFS) devices. FIGS. 19-22 show an exemplary kerf flap fixation device (KFFD). FIGS. 24A-B show an exemplary flap step fixation device (FSF). It should be noted that in Table 3 below, (FSF) is referred to as (BFSF). The results of this analysis are summarized herein in Table 3.

TABLE 3

Device Concept Evaluation Scoring Table

| Fixation Device Attribute | Importance (1 - Not important, 5 Very Important) | BFA | BFSF | BFW | CFFR (no screws) | CFFR (w/ screws) | CFS |
|---|---|---|---|---|---|---|---|
| Flap Holding Strength | 4 | 1 | 3 | 2 | 2 | 5 | 4 |
| Z-Axis Control & Alignment | 5 | 1 | 4 | 4 | 4 | 5 | 5 |
| X&Y Control & Alignment | 4 | 1 | 4 | 4 | 3 | 5 | 5 |
| Installation & Removal Usability/Speed | 4 | 5 | 5 | 5 | 3 | 2 | 3 |
| Adhesive Application Usability/Speed | 4 | 4 | 2 | 3 | 5 | 5 | 2 |
| Cost | 4 | 1 | 4 | 5 | 4 | 3 | 2 |
| Universal Application | 4 | 5 | 2 | 2 | 5 | 5 | 2 |
| | Raw Score | 19 | 24 | 25 | 26 | 30 | 23 |
| | Weighted Score | 24.9 | 32 | 33.3 | 34.6 | 40 | 30.9 |

As shown in Table 3, (BFW) scored higher in ease of installation and removal usability and speed, but scored poorly in flap holding strength and adhesive application usability and speed. (CFS) received lower scoring in adhesive application usability and speed, but scored higher than (BFW) in flap holding strength. (CFFR) scored highest of all the devices in adhesive application usability and speed, but got a low score in installation and removal usability and speed. (BFA) scored poorly in flap holding strength, z-axis control and alignment, and xand y-axis control and alignment. (FSF) scored low in flap holding strength and adhesive application usability and speed. (KFFD) is not listed in the Device Concept Evaluation Scoring Table, but it was the device overall favored by surgeons; it proved to be easy to install, uninstall, and relocate on the bone, and it fixated the flap successfully. As a result of these data, (KFFD) was selected to move forward with. Various desirable aspects of the devices not preferred by the surgeons were also refined and modified to be combined with (KFFD).

### Example 6: Investigation of Flap Fixation Devices

In this example, additional devices were investigated to determine which afforded the best overall properties for flap fixation. As described in Example 5, (KFFD) was selected for further development. Additional modifications to the overall design were sought, including decreasing the overall size, reducing the spring force required for fixation, reducing the rod/foot diameter for smaller kerf sizes, and making the parts of the device profile more ovular. After some iterations, the rotating foot flap fixation support (RFS) was developed, as shown in FIG. 29. Progression of this refinement from (KFFD) can be seen in FIG. 36. As can be seen in FIG. 36, a spring mechanism from the (CFS) fixation device was used to replace the screw fixation method originally used in the (KFFD). The spring component from (CFS) provided high z-axis control and alignment, and x- and y-axis control and alignment. The flexible loop and large base of (KFFD) were also ultimately abandoned in favor of a combination of the spring mechanism and foot fixation to hold the flap in place from the underside and the top. T-shaped hooked feet were tested but damaged live tissue and the design was quickly abandoned. Surgeons then tested this new device over several iterations and rated it highly. This new design was easy to install and remove with just one hand and axis alignment is adequate for intraoperative fixation.

The final design (RFS) comprises a thumb plunger with an indent which marks the position of the foot (F), a base (SB), which holds the flap in place from the top while the foot holds it from the bottom, and a spring (D), which has a spring constant (K) strong enough to hold the flap but not so strong as to damage any tissue. When the thumb plunger is pressed down the foot (F) extends to reach under the bottom of the flap to be fixated, wherein the indent on the top of the thumb plunger indicates the orientation of the foot. Releasing the thumb plunger engages the spring (D) and clamps the bone flap between the foot and the base (SB). The device can be easily removed by rotating the foot via the plumb plunger to disengage the spring and foot from the bone flap.

### Example 7: Investigation of Flap Fixation Devices

In this example, an additional device was investigated for flap fixation. As described in Example 5, several devices were explored. As described in Example 5, a cranial flap fixation support device (CFFS) was evaluated by surgeons and its various aspects refined according to surgeon preferences. The installation speed of said device is similar to the existing standard of care but takes longer relative to other fixation device aids as described herein. Said device also requires the use and removal of screws. (CFFS) comprises adequate holding strength for intraoperative fixation and is capable of resisting gravity if positioned correctly on the bone flap. The x-, y-, and z-axis control and alignment are also adequate for intraoperative fixation. The progressive iterations of (CFFS) can be seen in FIG. 34. The handling feature on the first version of (CFFS) was too short; in the second version the handling feature was sized well, but the device did not follow the skull contour and so the handle needed to be moved to the opposite side. The third iteration of (CFFS) had a good handling feature, but was not feasible on small flaps, and a later version shortened the handling feature and added vertical ribs for handling and decreased the boss ID to improve screw alignment.

The final version of (CFFS) comprised some features of (BFW) including (BFW)-style kerf ribs and 50 A durometer material to compress in the kerf. Key takeaways from surgeon evaluation were that the vertical kerf rib used works well and allows adjustments to be made with cam action, but noted that that the holding feature must be on the bone flap side, and the ribs do not go deep enough to engage all sizes of potential bone flaps. Although the surgeons were able to efficiently and accurately use (CFFS), it was not the device they ultimately preferred.

### Example 8: Investigation of Flap Fixation Devices

In this example, an additional device was investigated for flap fixation. As described in Example 5, several devices were explored. As described in Example 5, a cranial flap fixation device with retainer (CFFR) was evaluated by surgeons and its various aspects refined according to surgeon preferences. Surgeons found installation and removal to be difficult and time consuming due to the devices popping out as new devices are installed also, leading to a high “fiddle” factor. They also found it difficult to control and align the z-axis due to having to press down on the flap during installation without disturbing placement of previously installed devices. The next version of (CFFR) featured an increased arch radius to accommodate adhesive composition delivery and increase flexibility of the device. This final version still scored poorly in usability and removability and surgeons did not find the use of screws to be feasible with the use of a fast-setting adhesive composition. A version using pegs instead of screws scored higher on usability and removability, but poorly on flap holding strength. Variations on these versions could lead to a device that scores highly in usability and removability and flap holding strength.

### Example 9: Investigation of Flap Fixation Devices

In this example, an additional device was investigated for flap fixation. As described in Example 5, several devices were explored. As described in Example 5, a bone flap wedge (BFW) was evaluated by surgeons and its various aspects refined according to surgeon preferences. As described in Example 8 with (CFFR), surgeons found installation difficult and time consuming due to the devices popping out as new devices were installed and the z-axis was difficult to set and control due to having to press down on the flap during installation without disturbing placement of previously installed devices. FIG. 26 shows an exemplary (BFW) with varying durometer ribs. The first version presented to surgeons was a simple cross design with varying durometer ribs to be wedged into the kerf. Surgeons determined that said ribs and the handling feature were too small. (BFW) v.2 featured larger kerf filling features and a larger handling feature; however, the cylindrical shape of the device allowed the flap to rotate within the kerf space, which is not desired. (BFW) v.3 featured a rectangular profile to prevent the flap from moving, but the singular compressible kerf rib would not fit a 2 mm kerf. (BFW) v.4 provided 2 kerf ribs on either end of the device to fit multiple sized kerf spaces and featured a barrel profile for improved handling. However, the double-ended kerf rib design proved to make the device too top heavy. The final iteration of (BFW) still featured a double-ended kerf rib design but reduced the height to improve stability. This version of the device still had a problem with popping out when additional devices were placed but would be efficient and effective where only one fixation aid is necessary.

A final version (BFW) features a 2 mm spacer on one side of a low-profile base and a 3 mm spacer positioned on an opposite place as the 2 mm spacer on the other side of the low-profile base. The base features notches evenly spaced across the base for improved user handling, and the spacers are comprised of a 50 A durometer material.

### Example 10: Preparation of Adhesive Composition and Surgical Site for Cranial Flap Fixation

An exemplary adhesive composition, as described herein, may be used following a surgical procedure, e.g., a craniotomy, to fixate a bone flap in its desired position against the native bone. The adhesive composition may be used in conjunction with mechanical fixation aids as described herein. Once a surgeon completed a craniotomy or other procedure indicating the need for flap fixation, the site was prepared to receive the adhesive composition and fixation aids. The surgeon irrigated bone debris and any other tissue from the fixation/implantation site. The adhesive composition for cranial flap fixation comes in a sterile kit including 2 sealed mixing bowls containing the powder compositions for Composition 1I, as in Table 1B, 2 pre-filled aqueous syringes, 2 spatulas, 2 delivery syringes, 4 temporary fixation devices, and a burr hole mold, all nestled within an inner blister tray, wherein said inner blister tray is contained within an outer blister tray to maintain a field of sterility.

The cranial flap to be fixated was aligned and fixated with mechanical fixation aids prior to the adhesive composition being activated. Once the fixation devices were placed, the adhesive composition was activated and applied. Once the adhesive composition has been activated, it must be worked with quickly due to the fast setting-time. The surgeon opened one mixing bowl containing powdered bone adhesive, e.g., Composition 1I as in Table 1B, and carefully inject the liquid provided in one of the pre-filled syringes into the bowl. The surgeon mixed these components with the small spatula for approximately 30 seconds until it became a homogenous composition. The composition was loaded into the provided delivery syringe; this step should take no more than 30 seconds, for a total of 60 elapsed seconds thus far. The surgeon injected the adhesive composition into the flexible mold provided to make pre-formed disk(s) which are the appropriate size for the burr hole(s) created during the craniotomy. Said disks should be allowed to cure for at least 4 minutes and 30 seconds. Table 4, as seen below, shows the average time to be spent on each step, as well as the total elapsed time from first mix to final contour.

TABLE 4

Instruction Overview for Fixation and Cosmesis

| Step No. | Step | Total Elapsed Time from First Mix | Duration of Step |
|---|---|---|---|
| 1 | Activate and mix first dose | 30 sec | 30 sec |
| 2 | Load syringe | 1 min | 30 sec |
| 3 | Inject bone adhesive into flexible mold | 1 min 30 sec | 30 sec |
| 4 | Inject bone adhesive into kerf | 2 min | 30 sec |
| 5 | Contour | 2 min 30 sec - 3 min | 30 sec - 1 min |
| 6 | Remove fixation devices | 4 min 15 sec | 15 sec |
| 7 | Remove disk(s) from mold and place in burr hole(s) | 4 min 30 sec | 15 sec |
| 8 | Activate and mix second dose | 5 min | 30 sec |
| 9 | Load syringe | 5 min 30 sec | 30 sec |
| 10 | Inject bone adhesive over burr hole disk(s) | 6 min | 30 sec |
| 11 | Inject remaining bone adhesive around kerf | 6 min 30 sec | 30 sec |
| 12 | Contour | 7 min - 7 min 30 sec | 30 sec - 1 min |

### Example 11: Delivery of Adhesive Composition and Flap Fixation

An exemplary fixation device, as described herein, may be used in a surgery to temporarily fixate a flap in its permanent position during the delivery of an adhesive composition, with or without the need for removal. The steps disclosed in this example follow the actions disclosed in Example 10. The surgeon removed the soft tissue from the flap in the locations where mechanical fixation aids were to be placed. The surgeon applied 4 fixation devices to the bone flap by: holding the device in their fingers by the base (SB) and using their thumb to push on the top thumb pad, thus compressing the spring (D) and expanding the space between the foot (F) and the base (SB), wherein said space corresponds to the thickness of the flap; foot end (G) hooks under the bottom of the flap when spring (D) is compressed, and release of spring (D) applies force between the flap and the base (SB); base (SB) contacts the top surface of the flap and native bone such that when the spring (D) is released, the device fixated the flap in place against the native bone. Said fixation devices should be evenly spaced and the flap positioned to maintain equal width along the kerf space and stability while applying an adhesive composition. The surgeon used a provided delivery syringe preloaded with adhesive composition, as described in Example 10, and injected the material into and along the kerf space in an equidistant pattern around the entirety of the flap, while avoiding injecting material onto fixation devices or burr holes. The surgeon used the provided spatula provided to contour or trim excess material from around the kerf line. At this point in the procedure, no sooner than 4 minutes and 15 seconds from the time the adhesive composition was mixed together, the temporary fixation devices were removed. At this point, the pre-formed disks created out of the adhesive composition, as described in Example 10, were placed into the burr holes created during the craniotomy.

Once the surgeon placed the pre-formed hardened adhesive composition into the burr holes, a second dose of adhesive composition was prepared for a final fill of the kerf space and burr holes, and any needed cosmetic adjustments of the composition. Preparation and activation of the second dose of adhesive composition followed the same steps as described in Example 10. The surgeon activated the powder composition in the second mixing bowl by adding the liquid from the second pre-filled syringe and mixing the components into a homogenous blend. The homogenous material was then loaded into the second provided delivery syringe. The surgeon then injected the adhesive composition into the burr holes on top of the pre-formed disks of the same material. The surgeon also injected the material into and along the unfilled kerf space, layering the material as necessary, to completely fill any void space in the kerf. The surgeon used the provided spatula to contour the adhesive composition while it was in a putty state, e.g., up to 3 minutes and 30 seconds from activation, for final cosmesis to provide a smooth, flat transition surface between the native bone and the flap being fixated.

### Example 12: Cranial Flap Fixation Utilizing Rotating Foot Flap Fixation Support Device

An exemplary fixation device, as described herein, may be used in a surgery to temporarily fixate a flap in position while allowing spot or continuous injection of an adhesive composition with or without need for removal. As described in Example 5, an exemplary rotating foot flap fixation support device (RFS) was developed and preferred by surgeons. After the necessary procedure was performed, e.g., a craniotomy, a detached flap and kerf space were left. Four (RFS)s were presented to the surgical field and placed along flap surface to aid in fixation by: pushing down on the thumb plunger, compressing spring (D), which allows foot (F) to span the flap thickness (FE); and then rotating and releasing pressure on the thumb plunger to attach (RFS) to the flap by the force provided by the semi-loaded spring (D), wherein base (SB) contacts both the flap and native bone, thus fixating the flap. Once each (RFS) device was secured, the surgeon activated and delivered the bone adhesive composition, as described herein in Example 10, to the kerf space and burr holes created during the surgical procedure. Once the first dose of bone adhesive composition was prepared, the surgeon delivered the composition via syringe to the kerf space, while avoiding getting any of the adhesive composition on the (RFS)s or burr holes. The surgeon used the provided spatula to trim any excess composition from around the kerf line. At this point in the procedure, no sooner than 4 minutes and 15 seconds from the time the adhesive composition was mixed together, the (RFS)s were removed. Once the (RFS)s have been removed, the pre-formed disks created out of the adhesive composition, as described in Example 10, were placed into the burr holes created during the craniotomy.

After the surgeon placed the pre-formed hardened adhesive composition disks into the burr holes, a second dose of adhesive composition was prepared for a final fill of the kerf space and burr holes, and any needed cosmetic adjustments of the composition. Preparation and activation of the second dose of adhesive composition followed the same steps as described in Example 10. The surgeon activated the powder composition in the second mixing bowl by adding the liquid from the second pre-filled syringe and mixing the components into a homogenous blend. The homogenous material was then loaded into the second provided delivery syringe. The surgeon injected the adhesive composition into the burr holes on top of the pre-formed discs of the same material. The surgeon also injected the adhesive composition into and along the unfilled kerf space, layering the material as necessary, to completely fill any void space in the kerf. The surgeon used the provided spatula to contour the bone adhesive material while it was in a putty state, e.g., up to 3 minutes and 30 seconds from activation, for final cosmesis to provide a smooth, flat transition surface between the native bone and the fixated flap.

### Example 13: Cadaver Lab Flap Fixation Utilizing Rotating Foot Flap Fixation Support Device

An exemplary fixation device, as described herein, may be used in a surgery to temporarily fixate a flap in position while allowing spot or continuous injection of an adhesive composition with or without need for removal. A cadaver lab cranial flap fixation evaluation was performed using exemplary fixation devices as described in Example 5, e.g., for clinical testing of the procedure or components used herein. 64 craniotomies were carried out in 16 cadaver heads, with four bone flaps from each cadaveric skull being fashioned, including frontal, pterional, parietal, and parieto-occipito-temporal region flaps (craniotomy size 40, 70, 70, and 100 mm respectively). Each flap was first secured with standard cranial plates and screws, then biomechanically tested using either quasi-static compression (1 mm/min) or staircase impact (up to 60 J impact energy at increments of 6 J) to analyze mechanical strength. The plates and screws were then removed and the same bone flap was secured using an exemplary adhesive composition, as described herein, e.g., Composition 1I as seen in Table 1B.

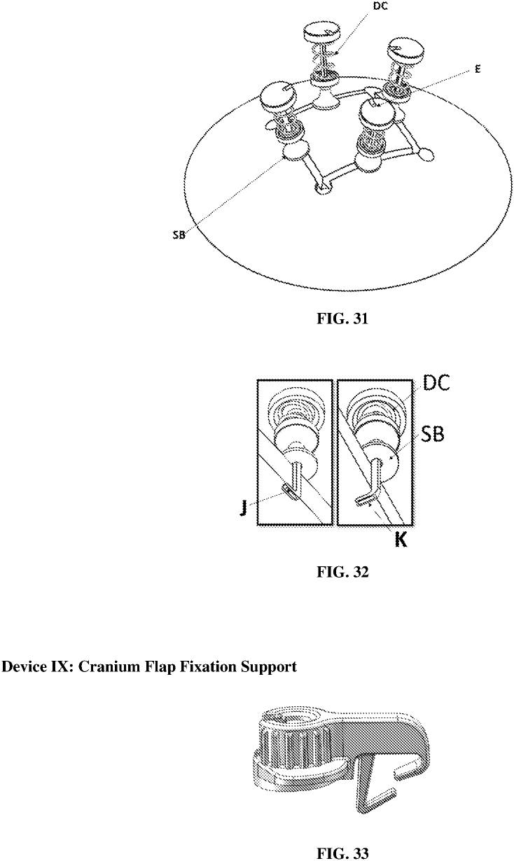

An exemplary rotating foot flap fixation support device (RFS), as described herein, may be used in a cadaver lab cranial flap fixation procedure. (RFS) may be used to temporarily fixate the cranial flap of the cadaver in position while allowing delivery of an adhesive composition. After a surgeon performed a craniotomy on the cadaver, a detached flap and kerf space were left. The series of (RFS) devices, as seen in FIG. 31, were presented to the surgical field and placed along the flap surface by: compressing spring (D) via the thumb pad allowing foot (F) to span the flap thickness, and then rotating and releasing pressure on the thumb pad to attach the device to the flap by the force provided by the semi-loaded spring (D) to the foot (F) and foot hook (G) which secures the device against the underside of the flap. There is an indent on the top of the thumb pad to indicate the position of the foot (F) when it is placed in the kerf. The space between the foot (F) and the base (SB) can accommodate a flap between about 5 mm and 10 mm thick.

Once each (RFS) device was secured, the surgeon activated and delivered the bone adhesive composition to the kerf space and burr holes created during the surgical procedure. Activation of the adhesive composition comprised the steps as described in Example 10. Once the first dose of adhesive composition was prepared, the surgeon delivered the composition via syringe to the kerf space, while avoiding getting any of the adhesive composition on the (RFS) fixation devices or burr holes. The surgeon used the provided spatula to trim any excess composition from around the kerf line. At this point in the procedure, no sooner than 4 minutes and 15 seconds from the time the adhesive composition was mixed together, the (RFS)s were removed. Once the (RFS)s have been removed, the pre-formed disks created out of the adhesive composition, as described in Example 10, were placed into the burr holes created during the craniotomy. Once the surgeon placed the pre-formed hardened adhesive composition into the burr holes, a second dose of adhesive composition was prepared for a final fill of the kerf space and burr holes. Preparation and activation of the second dose of adhesive composition followed the same steps as described in Example 10. The homogenous material was then loaded into the second provided delivery syringe. The surgeon injected the adhesive composition into the burr hole(s) on top of the pre-formed disks of the same material. The surgeon also injected the adhesive composition into and along the unfilled kerf space, layering the material as necessary, to completely fill any void space in the kerf. The surgeon used the provided spatula to contour the adhesive composition while it was in a putty state, e.g., up to 3 minutes and 30 seconds from activation.

These bone flaps were then tested under the same conditions as the mechanically secured bone flaps. A subset of these fixated flaps was also subjected to direct hydrostatic fluid pressure (up to 40 mm Hg at steps of 5 mm Hg, held for 5 secs at each step) to examine resistance to simulated CSF leakage prior to mechanical testing. Compared to metal fixation, fixation with an exemplary adhesive composition, as described herein, is approximately 41 times stiffer and 48 times stronger under quasi-static loading (stiffness 4216 ±1544 N/mm and peak force 5456 ±1920 N for TN vs. 103 ±81 N/mm and 113 ±89 N for metal hardware: n=32). At an impact energy of 6 J, cranial flaps fixated using metal hardware migrated to a peak deflection 13 times greater than specimens fixated with adhesive composition, and the residual plastic deformation was 30 times greater (peak elastic deflection 2.94 ± 0.81 mm and plastic deflection of 1.69 ± 0.93 mm for metal hardware vs. 0.24 ± 0.07 mm and 0.06 ± 0.04 mm for TN; n=31). Adhesive composition-fixated flaps did not show any signs of failure until subjected to an average impact energy of 27.48 ± 12.37 J at a peak force of 1093 ± 404 N (n=31). The adhesive composition did not fail at drop energies of 22.69 ± 13.95 J, recovering from peak deflections of 1.14 ± 0.84 mm at this energy level (n=32). Further, out of 16 specimens fixated with an exemplary adhesive composition tested under direct hydrostatic pressure; all of the specimens did not leak under pressures up to 35 mmHg held for 5 secs, and only 2 specimens leaked at 40 mmHg.

### Example 14: Safety Protocols and User Handling Guidelines During Flap Fixation Utilizing an Exemplary Fixation Device

An exemplary fixation device, as described herein, may be used in a surgery to temporarily fixate a flap in its permanent position during the delivery of an adhesive composition, with or without the need for removal. In this example, various safety protocols and user handling guidelines for flap fixation utilizing an exemplary fixation device, or series of devices, and an exemplary adhesive composition, as described herein. In this example, an exemplary fixation device is one as described in Example 5. The adhesive composition and exemplary fixation device(s), as described herein, are indicated for use to fixate a bone flap in its desired position following a surgical procedure or trauma that creates a bone flap to be fixated. The adhesive composition and fixation devices should not be used in the presence of any contraindication, which include but are not limited to:
Use in an actively infected site, where infections are suspected, or near an infection;
Use in areas where there has been previous surgery;
Use in pregnant women;
Use in patients with the following: abnormal calcium metabolism, metabolic bone disease, recent untreated infection, immunologic abnormalities, and systemic disorders which result in poor wound healing or will result in tissue deterioration over the implant site;
Use in patients who have not reached an age at which skeletal system growth is essentially complete; and
Use in patients who have undergone or are to undergo radiation therapy at or near the implant site.

When a surgeon does elect to perform a procedure, e.g., craniotomy, necessitating the post-operative use of an adhesive composition and exemplary flap fixation device, the following possible complications should be taken into consideration, which include but are not limited to:
Tissue thinning over the implant site;
Tenderness, redness, edema;
Seroma, hematoma, or infection;
Swelling or fluid collection;
Incompatibility or allergy, foreign body sensation, or neoplasm;
Swelling, flap sloughing, bleeding, local inflammation, bone loss, infection, or pain; or
Material fracture, material migration, flap mobility, aspiration of components, or fibrous tissue ingrowth.

Prior to use, the adhesive composition should be stored between 15-25° C. (59-77° F.). During use, the adhesive composition should be activated and used at between 18-22° C. (64.4-71.6° F.). Each dose of adhesive composition to be delivered via the provided syringe is 4 cc. It is important to work with the adhesive composition quickly once it is mixed, the working time of the adhesive composition from activation is approximately 3 minutes and 30 seconds, as can be seen in Table 4 in Example 10. The setting time of the adhesive composition before it fixates the flap is 10 minutes after delivery and contouring, during which the flap and adhesive composition should not be disturbed. If the operating room temperature or storage temperature prior to use is above 22° C., the adhesive composition will cure more quickly, shortening the working time of the adhesive composition. If the temperature is below 18° C., the adhesive composition will cure more slowly, extending the setting time.

When a surgeon does elect to perform a procedure, e.g., craniotomy, necessitating the post-operative use of an adhesive composition and exemplary flap fixation device, there are several precautions to consider. These are single use products and should not be reused or resterilized, and the fixation device(s) can only be implanted into a sterile field. Do not use the adhesive composition kit if the sterile barrier has been compromised. Do not use the adhesive composition kit if liquid or powder is visible outside its packaging before activation. Do not use the adhesive composition kit if the liquid or powder is spilled during activation or mixing. Do not prepare multiple doses of the adhesive composition at the same time, or combine doses. Do not attempt to reposition the flap after the end of the working time of the adhesive composition, 3 minutes and 30 seconds from activation of the adhesive composition, as can be seen in Table 4. Do not mix the adhesive composition with any other biomaterials. Do not use if the adhesive composition is expired. Contamination with lipid-based materials can interfere with the adhesive composition’s adhesion to bone and should be avoided. Exposure of the adhesive composition components to humidity prior to mixing can compromise the adhesive composition’s flap fixation strength or curing time. The exemplary fixation devices, as described herein, have not been evaluated in kerf lines greater than 3 mm, and should not be used in kerf lines greater than 3 mm. Fixation devices are able to accommodate neurocranium thicknesses up to 12 mm. A surgeon should take care to ensure the adhesive composition does not extrude beyond the intended application site, by carefully delivering the adhesive composition and contouring the adhesive composition during its working time. If the surgeon determines that revisional surgery is required, the exemplary fixation device(s) should be removed, and the surrounding bone should be reevaluated to ensure it is still viable. When a surgeon does elect to perform a procedure, e.g., craniotomy, necessitating the post-operative use of an adhesive composition and exemplary flap fixation device, it should be noted that failure to follow the provided instructions and consider the information and warnings provided herein may cause inadequate results or other unknown side effects.

1. A device for positioning or fixating a flap in or on a bone, wherein (i) the device holds the flap in proximity to the bone to allow for adhering the flap to the bone with an adhesive composition and (ii) the device does not comprise a bone screw, bone plate, connecting suture, or connecting wire.

2. The device of claim 1, wherein the device is part of an assembly of devices for positioning or fixating a flap in a bone.

3. The device of claim 1, wherein the flap is a cranial flap in a subject.

4. The device of claim 1, wherein the bone is the cranium of a subject.

5. The device of claim 1, wherein the device or assembly of devices is only used for a finite duration of time, e.g., is not permanently adhered to or implanted into a subject.

6. The device of claim 1, wherein the device or assembly of devices protrudes from the surface of the bone by about 1 mm, 2 mm, 3 mm, 5 mm, 7.5 mm, 10 mm, or more.

7. The device of claim 1, wherein the flap in the bone is surgically created or is created from trauma.

8. The device of claim 1, wherein the flap comprises native bone, xenograft bone, allograft bone, or a synthetic biomaterial.

9. The device of claim 1, wherein the device further comprises a biocompatible material comprising native bone, xenograft, allograft; or a synthetic biocompatible material.

10. The device of any one of claims 1-13, wherein the device comprises a synthetic biocompatible material.

11. The device of claim 1, wherein the synthetic biocompatible material comprises one or more of: steel or steel alloy, titanium or titanium-based alloy; a plastic material; an inorganic material such as hydroxyapatite, or alumina ceramics; organic materials such as transplanted bone, or organic composite; or combinations thereof.

12. The device of claim 1, wherein the device is of sufficient strength to withstand placement, adjustment and removal of said device, other devices, or flap without compromising the fixated position of the flap.

13. The device of claim 1, further comprising a means for allowing a user to position and fixate a flap, e.g., in its original or corrected position.

14. The device of claim 13, wherein said means is temporary or definitive.

15. The device of claim 13, wherein said means comprises a mechanical fixation feature.

16. The device of claim 1, wherein said device further comprises a mechanical feature usable within or across the kerf space dimension and native bone thickness.

17. The device of claim 1, wherein the device comprises a protrusion (e.g., wherein said protrusion may be pressed into the surface of native bone and/or flap using a mallet or other compressive methods to allow for temporary fixation of the flap in its original or corrected position).

18. The device of claim 17, further comprising a clamp comprising a supportive structure.

19. The device of claim 18, wherein the supportive structure comprises a compressive material.

20. The device of claim 19, wherein the compressive material comprises any soft durometer material or spring-like material, e.g., to allow for fixation of the flap in its original or corrected position.

21. The device of claim 1, wherein the device uses a compressive force (e.g., opposing compressive forces) to align and fixate the flap in its original or corrected position.

22. The device of claim 21, comprising vacuum suction forces for fixating the flap into its original or corrected position.

23. The device of claim 1, wherein the device further comprises a cam design (e.g., rotating cam design).

24. The device of claim 23, wherein said cam design engages compressive forces when rotated to temporarily fixate the flap in its original or corrected position.

25. The device of claim 1, wherein the device further comprises a durometer material, e.g., to apply compressive forces against the flap and bone to temporarily fixate the flap in its original or corrected position.

26. The device of claim 1, wherein the device further comprises a friction lock, e.g., to adjust alignment and lock the flap in its original or corrected position.

27. The device of claim 1, wherein the device further comprises a threaded screw, e.g., to adjust and lock the flap in its original or corrected position.

28. The device of claim 1, wherein said device creates a parallel surface between the flap and the native bone, e.g., to ensure the flap is in its native height and position.

29. The device of claim 1, wherein the device further comprises a structure to be bound to the bone via a screw, rod, plate, suction, or adhesive bonding.

30. The device of claim 1, wherein the device further comprises an adjustable Z-axis.

31. The device of claim 1, wherein the device further comprises a support structure.

32. The device of claim 31, wherein the support structure attaches to a section of the device from where a plurality of structures may be attached.

33. The device of claim 1, further comprising a screw relief hole.

34. The device of claim 33, wherein said screw relief hole comprises one or more sizes.

35. The device of claim 1, wherein said adhesive composition comprises a multivalent metal salt, an organic compound, and an aqueous medium.

36. The device of claim 35, wherein said multivalent metal salt is selected from the group consisting of calcium phosphate, tricalcium phosphate (e.g., alpha tricalcium phosphate or beta tricalcium phosphate), tetracalcium phosphate, and mixtures thereof.

37. The device of claim 35, wherein said multivalent metal salt comprises tetracalcium phosphate.

38. The device of claim 35, wherein said organic compound is selected from the group consisting of phosphoserine, carboxy ethyl phosphonate, phosphonoacetic acid, and mixtures thereof.

39. The device of claim 35, wherein said organic compound comprises phosphoserine.

40. The device of claim 35, wherein said aqueous medium is selected from the group consisting of water or a blood-based product.

41. The device of claim 35, wherein said aqueous medium is water.

42. The device of claim 1, wherein said adhesive composition is activated upon mixing the components with said aqueous medium (e.g., water).

43. The device of claim 1, wherein upon activation of the adhesive device with the aqueous medium, said adhesive composition has an initial tacky state for up to about 2 minutes after mixing.

44. The device of claim 1, wherein said adhesive composition during said tacky state has a separation strength of about 50 kPa to about 150 kPa after mixing.

45. The device of claim 1, wherein said adhesive composition has a putty state for about 8 minutes after mixing with the aqueous medium.

46. The device of claim 1, wherein said adhesive composition has a working time of about 7 to about 12 minutes after mixing with the aqueous medium.

47. The device of claim 1, wherein said adhesive composition has an adhesive strength upon curing of 250 kPa or greater.

48. The device of claim 1, wherein said adhesive composition comprises an additive, e.g., a salt, filler, viscosity modifier, an antibiotic, or other medication.

49. The device of claim 1, wherein said adhesive composition fixates the flap in its original or corrected position.

50. The device of claim 1, wherein said adhesive composition partially or fully fills the kerf.

51. The device of claim 1, wherein said adhesive composition seals the kerf, e.g., against leakage or infection.

52. The device of claim 1, wherein said device is engaged in methods to definitively fixate the flap, e.g., in its original or corrected position.

53. A method of fixating a flap (e.g., a bone flap), e.g., in its original or corrected position, by placing a device of any one of claims 1-52.

54. The method of claim 53, wherein said device comprises a means for delivering an adhesive composition.

55. The method of claim 53, wherein utilization of the device eliminates hardware protrusion.

56. The method of claim 53,, wherein eliminating of hardware protrusion reduces palpability and soft tissue irritation.

57. The method of claim 53, wherein said device further comprises a structural scaffold, e.g., to be placed in the osteotomy.

58. The method of claim 57, wherein said structural scaffold bonds to the flap and native bone.

59. The method of claim 53, wherein a plurality of devices is distributed into the kerf space (e.g., wherein said devices are placed partly within the kerf to provide roughly uniform separation between the edge of the flap and the cut edge of the native bone).

60. The method of claim 53, wherein said device is fixated into the kerf via protrusions, compression, screws, locking mechanisms and other such mechanisms.

61. The method of claim 53, further comprising use of an articulating arm, e.g., to manipulate the flap into its original or corrected position for definitive fixation.

62. The method of claim 53, wherein a bone substitute is delivered to the kerf space.

63. The method of claim 62, wherein said bone substitute is a biomaterial or combination of biomaterials.

64. The method of claim 63, wherein said biomaterial comprises an adhesive composition.

65. The method of claim 53, wherein said device comprises a malleable material, e.g., to allow manual adjustment of flap alignment.

66. The method of claim 53, wherein said device remains in the kerf space during delivery of the adhesive composition.

67. The method of claim 53, wherein said device remains in the kerf space during curing of the adhesive composition.

68. The method of claim 53, wherein said device is removable from the kerf space.

69. The method of claim 53, wherein said device remains in the kerf during placement of an additional device or additional treatment.

70. The method of claim 53, wherein said device remains in the kerf space such that an adhesive composition may be fully delivered into the kerf space creating a seal, e.g., against leakage.

71. The method of claim 53, wherein said device comprises a resorbable biomaterial.

72. The method of claim 71, wherein said resorbable material disperses and is replaced with host tissue over time.
